(19) United States
(12) Patent Application Publication
(10) Pub. No.: US 2012/0189706 A1
(43) Pub. Date: Jul. 26, 2012

(54) Macrocystis Pyrifera Derived Health and Wellness Products and Methods of Using the Same

(75) Inventors: Emmanuel Anthony Copp, Dallas, TX (US); Dale A. Glantz, San Diego, CA (US)

(73) Assignee: Knocean Sciences, Inc., Dallas, TX (US)

(21) Appl. No.: 13/440,020

(22) Filed: Apr. 5, 2012

Related U.S. Application Data
(63) Continuation-in-part of application No. 12/898,552, filed on Oct. 5, 2010, now Pat. No. 8,167,959.
(60) Provisional application No. 61/249,171, filed on Oct. 6, 2009.
(60) Provisional application No. 61/472,078, filed on Apr. 5, 2011.

(51) Int. Cl. A61K 36/03; A61K 31/737; A61P 31/14; A61K 35/56

(52) U.S. Cl. 424/522; 424/195.17; 514/54

(57) ABSTRACT

The exudate of macroalage is collected and selectively dried to produce a health supplement capable of oral, topical, or transdermal delivery to benefit the health of user. The macroalage is preferably brown macroalage of the species Macrocystis pyrifera and the exudate preferably collected within 12 to 24 hours of harvest. High purity Fucoidan derived from Macrocystis pyrifera is administered in an effective amount to benefit the health of a user. Collected exudate, dried exudate or High Purity Fucoidan may be combined with other materials such as krill oil to create a synergistic boost to Antioxidant power and health benefits.

### I. CROSS-REFERENCE TO RELATED APPLICATION

This application is a continuation-in-part of U.S. patent application Ser. No. 12/898,552, filed Oct. 5, 2010, which claims priority to U.S. Provisional Application No. 61/249,171, filed Oct. 6, 2009, the entire contents of which are incorporated by reference in their entirety. This application also claims priority under 35 U.S.C. §119(e) to U.S. Provisional Application No. 61/472,078 filed Apr. 5, 2011, the entire contents of which is incorporated by reference in its entirety.

### II. FIELD OF THE INVENTION

The present invention relates generally to products derived from the exudate of kelp as well as high-purity Fucoidan derived from harvested kelp, specifically the brown algae Macrocystis pyrifera. More particularly, the present invention relates to brown algae exudate and Macrocystis pyrifera derived pharmaceutical, nutraceutical, and cosmeceutical products and additives for oral, topical, and transdermal delivery to treat, prevent, or aid the health and wellness of an individual as well as the use of brown algae exudate and Macrocystis pyrifera high-purity Fucoidan as AntiOxidant additives for use with or in food, beverage, desert, or cosmetic products or in combination with other nutraceutical and cosmeceutical products to boost their final AntiOxidant impact.

### III. BACKGROUND OF THE INVENTION

The literature is fraught with tales of the health benefits of marine algae, and in particular, certain of the brown alga. Many of the perceived benefits are based on anecdotal evidence from the populations of the world that have included brown algae in their diets and products containing brown algae in their every day lives for centuries. Modern science has attributed most, if not all, of the purported health benefits to the Fucoidan found in brown macro algae.

Fucoidan is a type of complex carbohydrate, a sulphated polysaccharide, found in brown algae. The exact structure of Fucoidan differs depending on the alga from which the Fucoidan is originated, its growth environment, and its manner of extraction. One of the reasons is that compositions of fucose, galactose, xylose, glucuronic acid, mannuronic acid (normally associated with alginate distinctions) and the like, which are constituent components of Fucoidan, vary depending on the source algae and their growth environment. In addition, the positions of an ester bond and a glucoside bond on the constituent sugars may vary, further contributing to diversity of the structure of Fucoidan.

In any event, most prior artisans do not attribute any significance to the variation in the exact structures of the Fucoidans of different algal species and growing environments. Typically, the literature of the prior art accepts that the physiological properties of Fucoidans are more or less uniform across the board and its benefits, where appreciable, are based on the types of Fucoidan polymer molecules derived from the brown algae. Specifically, three types of active Fucoidan polymers are reported in the literature; 1) U-fucoidan, having about 20% glucuronic acid (U-fucoidan is widely believed to be active in carrying out cancer cell destruction); 2) F-fucoidan, a polymer of mostly sulfated fucose, and 3) G-fucoidan. The latter two polymers are believed to assist in restoring and repairing damaged cells, including skin cells, while all three are believed to boost the immune system individually or collectively. However, prior art products commercially available of these polymers only achieve a purity of 35% Fucoidan when derived from brown alga, such as Laminaria japonica.

Recent scientific studies have been commissioned to test the various potential benefits hypothesized to stem from the use or ingestion of Fucoidan. In the past decade, more than 600 studies have been added to the PubMed database relating to Fucoidan research. While the literature reports a wide spectrum of potential beneficial functions including potential anticoagulant and antithrombotic activities, a positive impact on the inflammation and immune systems, antiproliferative and antiadhesive effects on cells, and potential anti-viral, anti-inflammatory, anti-coagulation, and anti-tumor properties; the literature reports demonstrate modest, if any, statistically significant data on these potential beneficial functions. Likewise, the bulk of patent literature reports modest, if at all appreciable, synergistic effects when Fucoidan is used with other compounds having a known benefit. Prior literature does not recognize any benefit from use of the exudate, or as the present inventors call it “Kelp Oil”, expressed from the pores of kelp. In fact it is generally treated as a waste product. Similarly, concentrating the exudate to a powder by drying it to form what the present inventors call “Kelp Concentrate,” has never even been considered by prior artisans as discerned from the literature. Finally, the literature does not suggest any potential impact or synergy from the use of extremely High-Purity Fucoidan (meaning 90% or more) derived from dried, chopped and milled kelp, with or without the Kelp Oil removed.

While the prior art may present an invitation to experiment, there is no reasonable expectation of success in finding a High Purity Fucoidan-based solution, a Kelp Oil-based solution, or a Kelp Concentrate-based solution to the health, wellness and beauty concerns of today following the conventional wisdom in the art.

### IV. SUMMARY OF THE INVENTION

Various embodiments of the present invention provide health benefits far beyond the reasonable expectations of those skilled in the art armed with the contemporary knowledge of the state of the art. In addition, various embodiments of the present invention provide health benefits and precursors for products that strain, if not offend, the credulity in the art. Moreover, various embodiments of the present invention demonstrate an efficacy that far exceeds similar results for other brown algae. Finally, various embodiments of the present invention demonstrate a synergy and efficacy that satisfies a long-felt, yet unresolved need in the art to which most prior artisans had long since abandoned a solution.

The present invention is based, in part, on the discovery that “High-Purity Fucoidan” (meaning a purity of 75% or more) and Fucoidan compounds derived from the brown algae Macrocystis pyrifera differ not only in effect, but in kind, in the health and wellness benefits it provides to a human user. The present invention is also based, in part, on the discovery that the lubricious coating or exudate of kelp, referred to herein as its “Kelp Oil,” may be collected from any suitable kelp such as species of brown algae and used or further processed to form what is referred to herein as “Kelp Concentrate” and either or both used as a compound (or blended compound) for use in aiding, preventing, or treating a human condition, malady or effect. Separately, the “Kelp Oil” may also be a source of Fucoidan and/or Fucoidan based compounds also uniquely suitable for use in aiding, preventing, or treating a human condition, malady or effect.

The present invention marks the first time in history that a company, KNOCEAN Sciences, Inc., is making a product by isolating and refining the exudate of kelp. Most commercial processes using Macrocystis pyrifera, such as algin extraction, specifically wash away or reduce as much of the exudate as possible. In all prior art processes for making alginates or Fucoidan known to the inventors, the lubricious coating/exudate of kelp is not used. In prior art kelp commercial processes, the kelp fronds that are harvested from the ocean surface are the raw material processed into alginates or fucoxanthin and fucoxanthonol. By contrast, in the KNOCEAN process discussed herein the fucoxanthin and fucoxanthonol remain in the KNOCEAN's High Purity Fucoidan and other products.

While algin extraction is done using either dry or wet Macrocystis pyrifera, commercial Fucoidan extraction by firms in the industry, such as Marinova, Pty., is believed to be performed using only dry kelp. Similar to algin extraction, the Fucoidan extraction does everything possible to target only the Fucoidan such that any residual exudate/lubricious coating (e.g. “Kelp Oil”) that is not removed during the process would be an extremely small component if present at all in the final Fucoidan product.

By contrast, a key to a major feature of various embodiments of the present invention is the fact that the Kelp Oil is specifically targeted for capture and may be further processed to isolate its dried solids residue, the Kelp Concentrate, with everything else eliminated. As will be appreciated, this collection and selective drying of Kelp Oil goes against the conventional wisdom in the art.

According to the invention, an object of at least one embodiment of the invention is to collect the exudate/lubricious coating or “Kelp Oil” of a suitable macroalgae, preferably a suitable brown algae, and more preferably Macrocystis pyrifera, for use as an AntiOxidant source or health and wellness ingredient in nutraceutical, cosmeceutical and pharmaceutical applications and/or for inclusion in existing globally marketed products in foods, beverages, deserts, and cosmetics to boost their AntiOxidant content.

Another object of at least one embodiment of the invention is to harvest a suitable macroalgae, preferably a suitable brown algae, more preferably Macrocystis pyrifera, to extract and refine at least a portion of its cellular contents, the Kelp Concentrate, to use as an AntiOxidant for use as health and wellness ingredient in nutraceutical, cosmeceutical, functional foods, and pharmaceutical applications and significantly, for inclusion in existing globally marketed products in foods, beverages, deserts, and cosmetics to boost their AntiOxidant content.

Another object of at least one embodiment of the invention is to harvest Macrocystis pyrifera to extract its Fucoidan content and refine it to a purity of preferably greater than about 75%, and more preferably greater than 90%, using known providers such as Marinova Pty. of Australia to provide a “High Purity Fucoidan” product providing unexpectedly superior results referred to below.

At least one advantageous embodiment of yet another feature of the invention is to create a composition providing most, if not all, of the beneficial physiological results of the invention described herein by combining Macrocystis pyrifera derived Kelp Concentrate and Macrocystis pyrifera derived High Purity Fucoidan.

Yet another object of at least one embodiment of the invention is to provide a composition of matter comprising both Macrocystis pyrifera derived High-Purity Fucoidan and krill oil. The combination of the High-Purity Fucoidan and krill oil creates a physiological synergy for achieving some of the benefits of the invention as described herein.

Yet another object of a least one embodiment of the invention is to provide a composition of matter comprising both Macrocystis pyrifera derived Kelp Concentrate and krill oil. The combination of Kelp Concentrate and krill oil creates a synergy for achieving some of the benefits of the invention as described herein.

According to at least one advantageous embodiment of yet another feature of the invention, the Macrocystis pyrifera derived High-Purity Fucoidan, Kelp Oil and/or Kelp Concentrate is combined with krill oil to create a composition that creates a synergistic effect in achieving some, if not all, of the beneficial physiological results of the invention described herein, and in particular increasing the total AntiOxidant protection measured in Total ORAC5.0 values above their individual test scores.

According to at least one advantageous embodiment of yet another feature of the invention, Macrocystis pyrifera derived Kelp Concentrate is combined with probiotic products to create a composition for creating a synergistic effect in achieving some, if not all, of the beneficial physiological results of the invention described herein.

Yet another object of at least one embodiment of the invention is to provide a composition of matter comprising both Macrocystis pyrifera derived Kelp Concentrate and High-Purity Fucoidan with probiotic products to achieve a synergistic effect for achieving some of the benefits of the invention as described herein.

According to various advantageous embodiments of a feature of the invention, Macrocystis Pyrifera derived High-Purity Fucoidan or macroalgae derived Kelp Oil, or Kelp Concentrate provides beneficial anti-aging, healing, whitening, and/or regenerative properties to human skin when applied topically, transdermally, and/or taken orally. In accordance with this feature of the invention, in a presently preferred embodiment, the Fucoidan, Kelp Oil, and/or Kelp Concentrate is provided in the form of a cream or gel for massaging into the skin. In an alternate embodiment, the High-Purity Fucoidan or Kelp Concentrate is taken orally as a health and beauty supplement. In a most preferred embodiment, the Kelp Oil, Kelp Concentrate, and/or High-Purity Fucoidan is derived from Macrocystis pyrifera.

In another presently preferred embodiment, Kelp Concentrate is provided in beverages for sports nutritional purposes; in all juices such as orange, apple, pear, berry, and blends of juices; in all forms of dairy and soy milk products; in all forms of breakfast cereals, oatmeal, dark chocolate; and in all other candy like products, breads or bread equivalents, corn or flour based products including tortillas, cakes, and cookie products; and in non-food applications for oral hygiene including topical dental treatments, tooth pastes and mouthwashes.

According to at least one advantageous embodiment of another feature of the invention, the Macrocystis pyrifera derived High-Purity Fucoidan of the present invention provides anti-viral benefits (both prevention and treatment) that strain, if not offend, the credulity of those of ordinary skill in the art. In accordance with this feature of the invention, in one presently preferred embodiment, the High-Purity Fucoidan is provided in the form of an oral or transdermal agent for treating or protecting a person exposed to a virus or retrovirus such as HIV or herpes. In accordance with this feature of the invention, in another presently preferred embodiment, the High-Purity Fucoidan is provided in the form of an oil or gel providing protection from sexually transmitted diseases, such as HIV and herpes, during sexual intercourse by depositing it on or providing it with a condom or the like.

According to at least one advantageous embodiment of another feature of the invention, Macrocystis pyrifera derived Kelp Concentrate provides anti-viral benefits (both prevention and treatment) that strain the credulity of those of ordinary skill in the art. In accordance with this feature of the invention, in one presently preferred embodiment, the Kelp Concentrate is provided in the form of an oral or transdermal agent for treating or protecting a person exposed to a virus or retrovirus such as HIV or herpes. In accordance with this feature of the invention, in another presently preferred embodiment, the Kelp Concentrate is provided in the form of an oil or gel providing protection from sexually transmitted diseases, such as HIV and herpes, during sexual intercourse by depositing on or with a condom or the like.

These and other objects and advantages of the present invention may be realized by one or more of the embodiments or examples described herein.

Given the following enabling description, figures and examples, the novelty of the present inventions and the various respective advantageous features should become evident to a person of ordinary skill in the art.

### V. BRIEF DESCRIPTION OF THE DRAWINGS

FIG. 1 is a flow chart of a prophetic process for harvesting and processing the brown macroalgae Macrocystis pyrifera according to the invention.

FIGS. 2A-2C are tables of the composition of Macrocystis pyrifera.

FIG. 3 is a bar graph which illustrates the Oxygen Radical Absorption Capacity (ORAC5.0) of Macrocystis pyrifera derived products according to the invention as compared to existing products known to possess superior ORAC values.

FIGS. 4A and 4B are bar graphs of the IC50 of Macrocystis pyrifera Kelp Oil and 78% Fucoidan according to the invention.

FIGS. 5A and 5B are graphs of the AAPH induced cellular oxidation inhibited by Macrocystis pyrifera Kelp Oil and 78% Fucoidan experiments according to the invention.

FIGS. 6A,B-12A,B depict the Macrocystis pyrifera derived Fucoidan assay results for various viral strains.

### VI. DETAILED DESCRIPTION OF THE INVENTION

As alluded to above, the present invention is based, in part, on the discovery that High-Purity Fucoidan and Fucoidan compounds derived from the brown algae Macrocystis pyrifera differ not only in effect, but in kind, in the health and wellness benefits it provides to a human user. The present invention is also based, in part, on the discovery that the lubricious coating or exudate of kelp, referred to herein as its “Kelp Oil,” may be collected and used as a health and wellness product or further processed to its solid constituent, referred to herein as its “Kelp Concentrate,” and used as a health and wellness product.

While the Kelp Oil and Kelp Concentrate of any suitable brown algae or other kelp may provide some of the health and wellness benefits discussed herein, and should be understood to be part of the present invention, the present inventors contend that the Fucoidan, Kelp Oil, and Kelp Concentrate from Macrocystis pyrifera simply provide a unique set of physiological properties that are not expected by the conventional thinking and understanding of Fucoidan, Kelp Oil, and Kelp Concentrate in the art. The extraordinary and unexpected results from the Macrocystis pyrifera derived High-Purity Fucoidan products of the present invention simply defy and are contrary to the teachings and suggestions of the prior art.

In addition to the foregoing, the present inventors believe the synergistic effect of the Kelp Oil, Kelp Concentrate, and Macrocystis pyrifera derived High-Purity Fucoidan of the present invention are further enhanced and provide additional benefits when combined with krill oil (or a similarly effective material). Although not wishing to be bound by theory, the present inventors contemplate that krill oil provides not only a physiological synergistic effect but further enhances the efficacy of the present invention through its superior ORAC5.0 value and its ability to serve as an effective carrier of the active components of the Kelp Oil, Kelp Concentrate, and Macrocystis pyrifera derived High-Purity Fucoidan of the present invention.

I Macrocystis Pyrifera Collection and Processing

In a presently preferred prophetic embodiment, the assignee of the present application, KNOCEAN Sciences, Inc., (referred to herein as “KNOCEAN”), will obtain kelp for their various products from naturally occurring populations of giant kelp (Macrocystis pyrifera) off the coasts of California and Baja California, Mexico. Macrocystis pyrifera is perfectly suitable for commercial use because the surface canopy can be harvested several times a year without disturbing the submerged parts of the plant, where vegetative growth and reproduction occur. The surface canopy is continually regenerated by the rapid growth of young fronds.

KNOCEAN will obtain kelp using a combination of hand and mechanical harvesting techniques. For purposes of collecting Kelp Oil, Kelp Concentrate, and kelp for Fucoidan extraction, KNOCEAN will employ primarily mechanical harvesting techniques. As presently conceived, all mechanical harvests will be accomplished using specially designed harvesting vessels. These harvesting vessels can either be specifically designed or existing vessels that will be converted into harvesters by retrofitting the vessels. Vessels built from scratch can be engineered to hold from a few to several hundred tons of kelp if required for operations.

The vessels will be engineered to incorporate a specially designed retractable harvesting apparatus and kelp holding bin with a drainage system that efficiently diverts, screens, and captures in tanks the Kelp Oil that the plants naturally exude. These vessels will also have a built in crane or davit system as well as highly flexible ballast systems to maintain trim during the loading process. Other equipment may be installed on the vessel to chop or mill the kelp after it is brought onboard to help reduce or eliminate the need to process the kelp at land based facilities. KNOCEAN will initially do all additional kelp processing such as chopping and milling at their land based facilities. The kelp load capacity of KNOCEAN's harvesting vessels are anticipated to range from 10 to 50 tons.

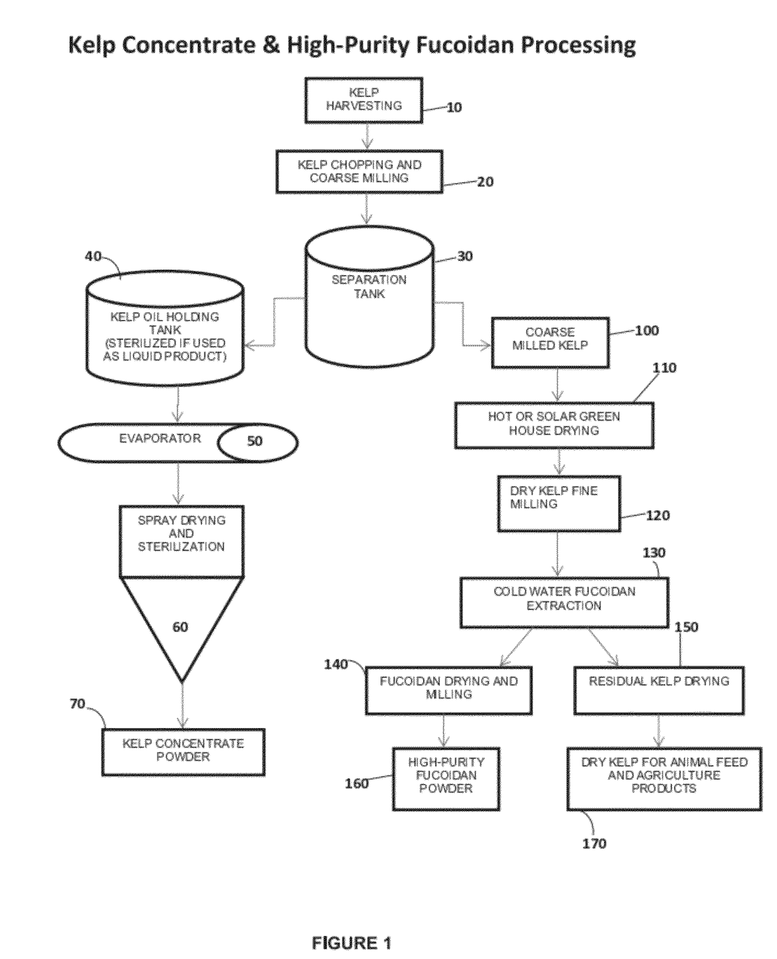

As presently conceived and depicted in FIG. 1, in a harvesting step 10, once the harvesting vessel reaches its designated kelp bed, the specially designed harvesting apparatus or draper system is rolled from the vessel's deck and lowered into the water. The vessel then slowly moves through the kelp bed's surface canopies and begins the harvesting operation. The harvesting apparatus is essentially a cutting rack that incorporates self-sharpening reciprocating blades mounted at the base of a conveyor system. Harvesting drapers can be mounted on either the bow or stern of the vessel depending on the design of the harvester. Whether bow or stern mounted, the draper system is lowered into the water to a depth of not more than four feet (per California's Code of Regulations). Once the reciprocating blades have cut the kelp, loose fronds are prevented from floating away by vertically rotating, toothed rollers. Guided by the rollers, the fronds move up the conveyer system and are deposited directly into netted cargo bags that have a 1-2 ton capacity. Once a bag is filled with freshly harvested kelp, it is repositioned in the bin of the boat utilizing the onboard crane. This bag filling and repositioning process is continued until the bin is completely loaded with filled bags of fresh kelp. KNOCEAN's vessels will be able to gather their 10-50 ton loads in anywhere from four to eight hours depending on the density of the canopies in the kelp bed being harvested.

Throughout the harvesting process and during the vessel's trip back to port, the freshly harvested kelp in the bin will continuously exude Kelp Oil from its pores and coats the fresh kelp that is brought onboard the harvesting vessel. The process of cutting the kelp also liberates additional Kelp Oil. The seawater water that comes on board with the kelp will quickly begin to drain off. The Kelp Oil that coats the kelp is much more viscous than seawater, so it drains off much slower than the seawater. The kelp harvester will preferably be designed to separate the majority of the sea water from the Kelp Oil. This may be accomplished by designing the shape or incline of the holding bin to drain liquids to specific screened locations. Once the liquid passes through the screens, it drains into a collection or piping system that can be either diverted overboard or into below deck holding tanks. The initial seawater drainage can simply be diverted overboard. Once the entire kelp load is obtained and most of the seawater has drained off, then the Kelp Oil can be collected by diverting the piping system entirely to the below deck holding tanks. This Kelp Oil collection process can continue from shortly after the kelp load is obtained until the vessel arrives back at the docking facility and the kelp bags are unloaded. The Kelp Oil collected during this process can then be removed from the below deck storage tanks through either an onboard or dockside pumping system.

When the vessel reaches port, the full cargo bags of freshly cut kelp can be unloaded by a crane directly into dockside kelp processing equipment or into specially designed trucks if the processing facility is located at a different site. If a truck is utilized, then it will be watertight and outfitted with a crane and kelp oil capturing system. Similar to the vessel, any kelp oil that continues to drain off the kelp while in the truck's bin is diverted through screens and a piping system to tanks mounted on lower sides of the truck. Any Kelp Oil collected in these holding tanks can be drained off using gravity or pumps similar to what would be used for removing Kelp Oil from the vessel's holding tanks. The truck is designed to haul approximately 20 tons of kelp and 200 gallons of Kelp Oil. The Kelp Oil that is captured on the boat is pumped from the vessel into totes or other containers for transport along with the netted fresh kelp by truck to the nearby processing facility.

At the processing facility, as again depicted in FIG. 1, the full cargo bags of fresh kelp are unloaded using the crane on the transport truck. The cargo bags are designed to easily release the kelp contents from the bottom of the bag into a large v-shaped hopper. The hopper then feeds the kelp into a large shredding machine to chop the kelp into approximately one inch pieces in a chopping/milling step 20. The chopped kelp can then be conveyed to a mill that grinds the kelp into pieces approximately one-quarter inch in size. The milled fresh kelp is then either pumped or conveyed to large separation tanks where the kelp is held for approximately 12 to 24 hours to allow the Kelp Oil that has been liberated by the shredding and milling process to accumulate by gravity at the bottom of the separation tanks in a separation step 30. The process of cutting and milling the kelp liberates substantial quantities of Kelp Oil. The 12 to 24 hour Kelp Oil accumulation period has been determined through testing to produce the highest quality Kelp Oil that maintains end-product functionality. At 12 to 24 hours, the accumulated Kelp Oil is drained from the separation tanks and pumped into Kelp Oil holding tanks in step 40. The Kelp Oil that was captured on the harvesting vessel and transport truck is also pumped into these same holding tanks. The Kelp Oil at this stage has a solids content of approximately 4 to 7%.

In an evaporation step 50, the Kelp Oil is pumped from the holding tanks into a thin film evaporator, a wiped film evaporator, sequential evaporators, or similar mechanical equipment that pre-concentrate the solids in the Kelp Oil to approximately 25 to 35%. Increasing the solids content of the Kelp Oil to this range increases the viscosity of the Kelp Oil from about 50 centiPoise (cP) to approximately 200 to 400 cP. This viscosity range significantly improves spray drying efficiency and particle size control of the finished powdered product. Pre-concentrating the solids beyond 35% results in the Kelp Oil developing crystalline material that would negatively impact the spray dryer's operation and efficiency. This additional discovery of the optimal process for Kelp Concentrate/spray drying results was another discovery made during testing.

In a spray drying step, the 25 to 35% solids pre-concentrated Kelp Oil is pumped to a spray dryer that has been specially designed for turning the concentrated Kelp Oil into KNOCEAN's Kelp Concentrate, which is the free-flowing powdered residue of the Kelp Oil. The spray dryer is designed to optimize the efficiency and cost effectiveness of converting concentrated Kelp Oil into the powdered Kelp Concentrate with the desired particle size without impacting product functionality or ORAC values. The optimal spray dryer configuration to obtain the highest functionality and ORAC value is feeding the Kelp Oil from an ambient temperature to a spray dryer inlet feed temperature of 180° C. and an outlet temperature or 90° C. To improve the cost effectiveness of the spray drying process, the inlet/outlet feed temperatures can be increased to 225° C./105° C. with only a minimal loss of functionality and ORAC Value (approximate 10% decrease) of the final product. These temperature configurations are believed to sterilize the final product sufficiently for cosmetic, supplement, and food ingredient applications, but if other uses require more onerous specifications, then an additional sterilization step or unit can be incorporated before or after the spray drying process. This added sterilization step would use flash pasteurization in the Kelp Oil stage or some other form of sterilization such as steam sterilization of the powdered Kelp Concentrate. Flash pasteurization of the Kelp Oil would be incorporated in the process for products that use Kelp Oil in its liquid state.

The resultant Kelp Concentrate from the sterilization and spray drying process might go through an additional fine milling step 70, depending on the customer requirements for the end application of the Kelp Concentrate. The appropriate particle sized Kelp Concentrate is then packaged into lined paper bags or fiber drums for transport to our end-product manufacturing partners or customers.

As presently conceived, the chopped and milled fresh kelp that is left over from the Kelp Oil separation process is handled separately to produce the dried kelp raw material that will be converted into KNOCEAN's High-Purity Fucoidan in a loading step 100. The chopped and milled fresh kelp is dumped from the separation tanks and loaded by means of a conveyor system into watertight end dump trucks that transport the kelp from the Kelp Oil and Kelp Concentrate processing facility to an inland drying location. The chopped and milled fresh kelp can be dried in a drying step 110 using a number of techniques including geothermal drying, wind drying, solar drying and mechanical drying. The most cost effective drying technique in California and Baja California, Mexico is solar drying. The best solar drying would be in a location at least 10 miles inland from the coast. An inland location is better since it will reduce or eliminate the coastal marine layer effect and result in decreased drying times.

Based on tests performed by KNOCEAN personnel, solar kelp drying is best accomplished on a concrete or thick black plastic drying surface. Prior to dumping the kelp pieces from the transport trucks, the concrete or plastic surface is cleaned with a commercial street sweeper or other cleaning device. This reduces the amount of extraneous material that could get into the final dried product. The kelp pieces are dumped into long rows on the concrete or plastic surface and then spread by hand using a rake or with a skip loader or similar vehicle equipped with a rake. The kelp pieces are spread over the concrete or plastic surface to a thickness of about 1-3 inches. The kelp is turned at least once a day during the drying process using a hay rake or by hand. The kelp is dried to 85-90% solids. The amount of time required for the kelp to reach the target of 85-90% solids generally ranges from 2-6 days depending on the drying location, season, and weather conditions. As will be appreciated, the kelp dries faster in hot dry desert conditions. Desert conditions reduce the overall drying time by about one day. On average, it takes 2-3 days to dry the kelp during the summer, 3-4 days in the late spring and early fall, and 5-6 days in the early spring and late fall. Once the kelp is dried, a skip loader or worker scoops it up and dumps or sends it into a hopper that feeds directly into a milling system in a milling step 120. The kelp should be processed and fine milled relatively soon after reaching the target dryness to prevent over drying and reduce quality of the end product. Preferably, the Fucoidan content of the kelp is extracted at this step 130 in the process.

The dried kelp pieces can be milled to a mesh size that is appropriate for whatever the finished product will be whether it is for High-Purity Fucoidan extraction in step 140, or animal feeds, aquaculture feeds, and/or agricultural applications in step 150. A mesh size of 40 is most suitable for the High-Puity Fucoidan extraction process currently being employed. If there are minimal kelp losses during the drying process, we would expect a recovery factor from wet to dry milled kelp somewhere in the 7:1 range. This assumes a wet kelp solids content of 12.6% and a dry kelp solids content of 85.0%. After milling, the dried kelp powder is then packaged in a packaging step 160 for shipment to KNOCEAN's High-Purity Fucoidan manufacturing partner, Marinova Pty. of Australia for the next stage of processing or packaged in a packaging step 170 into other final products. Depending on the next processing stages, the kelp could be packaged into fiber drums, plastic lined bags, supersacks, or other containers. Use of the dried kelp staring material described above results in the High Purity Fucoidan of the present invention when the dried kelp is subjected to the Fucoidan extraction process of Marinova Ltd. of Australia.

II Macrocystis Pyrifera Chemical Composition

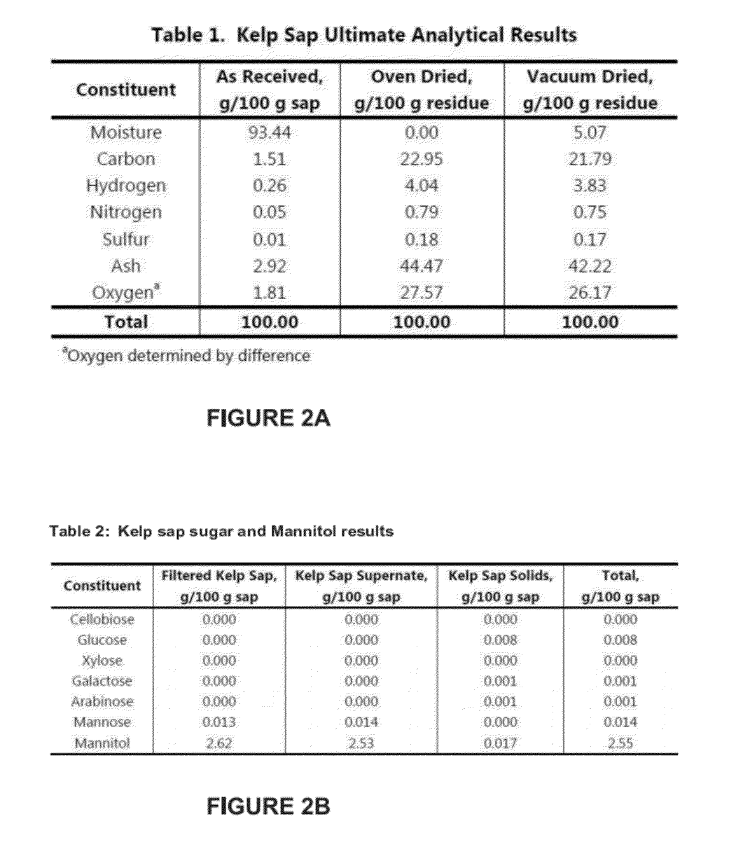

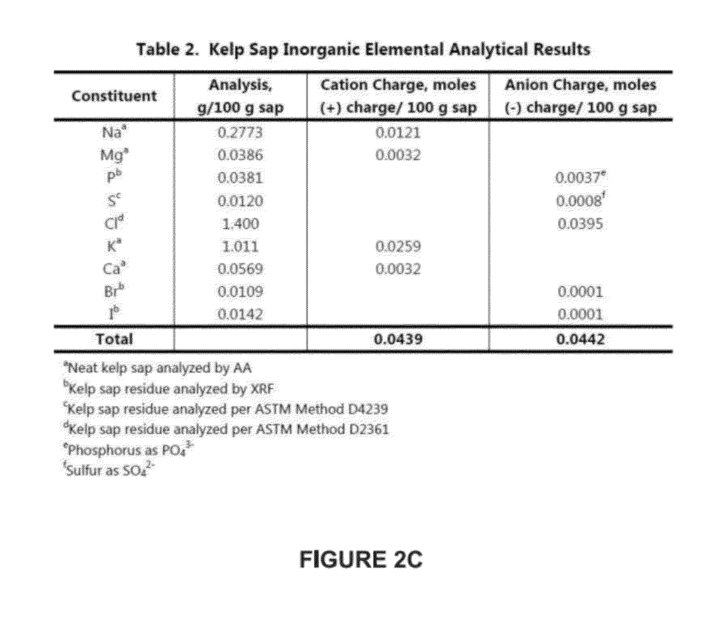

FIGS. 2A-2C are tables showing the chemical composition of Macrocystis pyrifera. As discerned from FIGS. 2A-2C, it is understood that Macrocystis pyrifera has a unique molecular weight and distribution of Fucoidan than other brown algae:

˜22.5% by weight <5,000 Daltons

˜40% by weight <20,000 Daltons

˜56% by weight <60,000 Daltons (assuming ˜50% by weight <50,000 Daltons)

The lower molecular weight fractions of Fucoidan, viz. <60,000 Daltons, more preferably <40,000 are perfectly sized for absorption of the native Fucoidan through the gastro-intestinal (GI) tract. Other brown algae Fucoidans are too large for absorption, or have been treated, e.g., hydrolyzed, de-polymerized, or otherwise reduced to smaller fragments, that while sized to be absorbed during digestion, have somehow been compromised in their native efficacy (if any). Human health conditions such as cancer (anti-tumor activity), diabetes (suppression of blood glucose level increase), thrombosis (anticoagulant effects), heart conditions (suppression of triglyceride and cholesterol level increase), anti-oxidative activity, anti-ulcer effects and enhancement of immunity are all believed to be aided by the ability to use of the native Fucoidan of Macrocystis pyrifera in the present invention.

Without wishing to be bound by theory, the present inventors believe that the preservation of the native pattern of sulfation and the integrity of the other structural features of the Macrocystis pyrifera Fucoidan plays a role in the synergy and extraordinary health benefits of the present invention. This role in the synergy is also believed to be true for the higher molecular weight fractions that lend themselves to applications wherein there is no requirement of low molecular weight, e.g. topical application, skin rejuvenation/antiaging benefits, anti-coagulant/anti-thrombotic benefits, etc.

Macrocystis pyrifera also has a unique L-Fucose:D-Galactose ratio in Fucoidan. The high ratio itself does not suggest or extrapolate to a given benefit, but the present inventors believe that the unique ratio and prevalent Fucose structures lead to more favorable chain conformations useful in treating certain conditions (e.g. thrombus).

Macrocystis pyrifera also has a unique high sulfite content in High-Purity Fucoidan. The present inventors contend that the bio-activity of High-Purity Fucoidan improves with increasing “native” and sulfite content and when combined with the other attributes of Macrocystis pyrifera derived Fucoidan plays a role in the synergy/co-action that results in the incredible health benefits of the present invention. The Kelp Oil and Kelp Concentrate derived from harvested Macrocystis pyrifera, also provides similar chemical structure and organic and inorganic content, to explain its extraordinary enhancement to AntiOxidant power.

Macrocystis pyrifera also has unique linking of monomer units that create a structure with enhanced ligand-like properties that enable binding. The sulfite group (and hydroxyl, acetyl) attachment and positioning on the monomer rings (2, 3 and/or 4 positions resulting in equatorial or axial distribution of active sites) are also believed by the present inventors to be a key to the Macrocystis pyrifera derived High-Purity Fucoidan's bio-activity and the incredible health benefits that are not found or achievable with other Fucoidans. This feature and its combination with the other unique features of Macrocystis pyrifera derived High-Purity Fucoidan are believed to explain, in part, the incredible test results set forth herein that demonstrate that the High-Purity Fucoidan of the present invention is not only an effective binder and blocker in case of some viruses, but that in the case of Dengue and HIV viruses, the High-Purity Fucoidan is vastly superior to the best known treatments for these ailments.

In addition, Macrocystis pyrifera derived High-Purity Fucoidan, separately Kelp Oil and Kelp Concentrate, has a unique polyphenolic content that the present inventors believe contributes to its incredibly high antioxidant benefit, and in particular, singlet oxygen quenching which lends itself to the skin rejuvenation and whitening aspects of the present invention. Again, not wishing to be bound by theory, the present inventors theorize that the high molecular weight Fucoidan (preferably >40,000) component may serve as a carrier of polyphenols and provide other antioxidant benefits (e.g. hydroxyl radical quenching). Notably, as a brief aside, the Kelp Oil and Kelp Concentrate of Macrocystis pyrifera provides superior (even more superior than its High-Purity Fucoidan) anti-oxidative benefits with its polyphenolic content contributing to singlet oxygen quenching, thereby leading the present inventors to a presently preferred embodiment of the invention that uses Kelp Oil or Kelp Concentrate for cosmeceutical skin rejuvenation, whitening, and anti-aging products. The composition of Kelp Concentrate and High Purity Fucoidan, in contrast to vitamins, includes other nutrients in recognized plant-based foods that promote health. These other nutrients are increasingly known as phytonutrients and are compounds in plant-based foods that play a potentially beneficial role in the prevention and treatment of disease. The present inventors believe polyphenol phytonutrients (flavonoids) in Macrocystis pyrifera promote health through their bioactive intensity with lower molecular structures resulting in more rapid resorption into the blood stream to deal with Free Radicals. Acting in a primary role as a more effective AntiOxidant engine, in contrast to other AntiOxidants such as vitamins which are on the lowest scale of ORAC values, the present inventors also believe the kelp compounds have a linked benefit by their hydrophilic-water soluble AntiOxidant characteristics making their role significant in providing positive features beyond ORAC scale, and that is in supporting angiogenesis, the balance in the body of blood vessels by promoting factors that reduce antiangiogeneses results, such as blood vessels that feed tumors or other diseases. Relating to the growth of new capillary blood vessels in the body, angiogenesis is an important natural process in the body used for healing and reproduction. The body controls angiogenesis by producing a precise balance of growth and inhibitory factors in healthy tissues. When this balance is disturbed, the result is either too much or too little angiogenesis. Abnormal blood vessel growth, either excessive or insufficient, is now recognized as a “common denominator” underlying many deadly and debilitating conditions, including cancer, skin diseases, age-related blindness, diabetic ulcers, cardiovascular disease, stroke, and many others.

The present inventors believe that the Kelp Concentrate and High Purity Fucoidan promotes health by both its ORAC5.0 anti-Free Radical impact and also its

antiagiogeneses factors, potentially in combination with products available now and under development to include new vascular health products, to provide for the growth of new healthy blood vessels thereby restoring the body's blood vessel balance.

III Macrocystis Pyrifera and its Unexpectedly Superior Antioxidant Properties, ORAC Value, and Synergistic Effects

A. Antioxidants and ORAC Value

When discussing the benefits of “antioxidants,” the potency of products are typically based on, or extrapolated from, their “ORAC” value. “ORAC” is an acronym for “Oxygen Radical Absorption Capacity.” The Brunswick Labs original ORAC test measured antioxidant capacity against the peroxyl radical. ORAC has become the accepted standard of antioxidant measurement in the industry. Previously, most known published numbers on ORAC values were limited to this narrow peroxyl radical activity. However, in addition to peroxyl, the primary radicals include hydroxyl, peroxynitrite, singlet oxygen, and superoxide anion. Each is formed differently and behaves differently in human metabolism. To get a more comprehensive, unified system of testing antioxidant capacity, the Total ORAC5.0 tests are used to get values against all five free key radicals. Total ORAC5.0 provides both (1) results against each of the 5 free individual radicals, and (2) a single, aggregate Total ORAC result. The individual components of ORAC5.0 are:

ORAC is for the peroxyl radical and its score;

NORAC is for the peroxynitrites radical and its score;

SORAC is for the superoxide anions and its score;

HORAC is for the hydroxyl radicals and its score;

SOAC is for the singlet oxygen radicals and its score.

These individualized measures were created as experimental evidence suggested that there are six major reactive oxygen species (ROS) causing oxidative damage in the human body. These species included: superoxide anion (O2.-); hydrogen peroxide (H2O2); peroxyl radicals (ROO.); hydroxyl radicals (HO.); singlet oxygen (1O2); and peroxynitrite (ONOO—). The peroxyl radical is the most abundant free radical in the human body. Another one of the more relevant radicals in biological regulation is superoxide anion radical. The superoxide anion is formed by the reduction of molecular oxygen in the process of energy metabolism.

Accumulated evidence indicates that reactive oxygen species, such as peroxyl radicals (ROO.), hydroxyl radicals (HO.), the superoxide anion (O2-.), and singlet oxygen (1O2), are involved in the pathophysiology of aging and a multitude of diseases. To counteract the damage of the reactive oxygen species on living cells, a defense system is designed biologically to neutralize the reactive oxygen species or to prevent the reactive oxygen species from being generated in the first place. Depending on the reaction mechanisms, antioxidants are often classified into two major categories: radical chainbreaking antioxidants and preventive antioxidants. Chainbreaking antioxidants convert reactive free radicals (e.g., HO.) to stable and thus nonaggressive molecules through hydrogen atom transfer reactions between HO. and the antioxidants. As a result, the autoxidation chain reactions between the free radicals and the cellular molecules are terminated. Preventive antioxidants inhibit the oxidation reaction from occurring by either converting the precursors of the reactive oxygen species to unreactive species or inhibiting the oxidation reaction. To counteract the assault of the superoxide anion reactive species, living cells have a biological defense system

It is evident that the antioxidant defense “team” in living cells contains individual antioxidants that function in very different tasks in the battles against oxidative stress and reactive oxygen species. Although there has been a validated assay for peroxyl radical absorbance capacity (ORAC) (1-5), no such assay had been developed for any other of the reactive oxygen species, until Brunswick Laboratories first published methods for the analysis of antioxidant capacity using the hydroxyl, and superoxide anion radicals. Brunswick Laboratories has subsequently addressed the other oxygen radical species [singlet oxygen (1O2); and peroxynitrite (ONOO—)] by developing assays that assess their contribution to total antioxidant capacity.

In looking at various fruits and vegetables, Brunswick found that there is little or no correlation among the different radical sources used to assess antioxidant capacity. One example that stands out is with tomatoes, which has a very low antioxidant capacity as measured with the peroxyl radical, but much higher antioxidant capacity using the singlet oxygen radical. Furthermore, the original peroxyl ORAC represents no more than 27% of the antioxidant potential of selected fruits and vegetables. The other radical assays added to the ORAC suite represent the preponderance of antioxidant potential.

By measuring all primary reactive oxygen species, ORAC5.0™ provides new opportunities which can be used in the formulation of nutritional products that deliver quantifiable, maximal protection against multiple radical sources. The present inventors believe KNOCEAN is the first to provide detailed AntiOxidant capacity for seaweeds, and specifically Macrocystis pyrifera to indicate the relative direction of each specific benefit for the skin free radical protection (peroxynitrites {NORAC} and singlet oxygen {SOAC}) and for internal to the body free radical protection (hydroxyl {HORAC}, peroxyl radicals {ORAC} and superoxide anions {SORAC}).

B. The Health Benefits of Antioxidants

A “free radical” is an unstable molecule with an unpaired electron. In a process called oxidation, the unpaired electron steals electrons from other molecules, creating new unstable free radicals. Sometimes free radicals are called oxidants because they cause the oxidation process. Free radicals occur naturally in the body but can be increased by environmental and lifestyle factors, such as stress, pollutants or poor diet, and other substances, such as nicotine or alcohol. In the oral cavity, dental procedures and materials such as bleaching agents, dental cements and composite fillings can also increase the level of free radicals.

Antioxidants are molecules that counteract the process of oxidation. The large, complex antioxidant molecules can bond with the unpaired electrons of free radicals, effectively neutralizing the oxidation process. Some of the most effective antioxidants come from fruits and vegetables; dietary antioxidant supplements are also available. An emerging and exciting means of countering the effects of free radicals is topical antioxidants, which are applied and not ingested. Research has already proven the effectiveness of topical antioxidants on skin cells. New research is demonstrating that combinations of antioxidants can be applied topically to oral cells to neutralize free radicals in oral tissues.

Rather than provide a long explication of the perceived health benefits of antioxidants, suffice it for purposes of the present application to generalize that antioxidants ingested in the body are thought to boost the body's immune system, while antioxidants applied to the skin are thought to provide anti-aging, healing and rejuvenation benefits.

A lesser known potential health benefit of the antioxidant properties of the present invention stems from helping good oral hygiene. To be more specific, oral infection and periodontal disease have been identified as risk factors and studies published by the New England Journal of Medicine and Journal of the American College of Cardiology affirm the link between periodontal disease and vascular disease, including heart attack and stroke.

When there are too many free radicals, or oxidants, in the body, the imbalance is called oxidative stress. In the oral cavity, oxidative stress is associated with infection or inflammation of the gums (gingivitis) and other soft tissues (periodontitis). But factors including alcohol consumption, exposure to nicotine, dental procedures, bleaching agents, dental cements and composite fillings also lead to oxidative stress. And oxidative stress in the oral cavity can be a major contributor to systemic oxidative stress—which leads to chronic diseases, such as rheumatoid arthritis or vascular disease including heart attack or stroke.

C. ORAC Experimental Results

Fucoidan Extract (78% Purity) of Macrocystis pyrifera, Powder

| Results: Test | Result | Units |
|---|---|---|
| Antioxidant power against peroxyl radicals | 68 | μmole TE/gram |
| Antioxidant power against hydroxyl radicals | 152 | μmole TE/gram |
| Antioxidant power against peroxynitrite | 1 | μmole TE/gram |
| Antioxidant power against super oxide | 31 | μmole TE/gram |
| anion | | |
| Antioxidant power against singlet oxygen | 62 | μmole TE/gram |
| Total ORAC5.0 (sum of above) | 314 | μmole TE/gram |

Kelp Oil Liquid, Total Phenolics 0.74 mg/Milliliter

| Results: Test | Result | Units |
|---|---|---|
| Antioxidant power against peroxyl radicals | 10.86 | μmole TE/ |
| | | milliliter |
| Antioxidant power against hydroxyl radicals | 47.00 | μmole TE/ |
| | | milliliter |
| Antioxidant power against peroxynitrite | 0.49 | μmole |
| | | TE/milliliter |
| Antioxidant power against super oxide | 3.78 | μmole |
| anion | | TE/milliliter |
| Antioxidant power against singlet oxygen | 43.09 | μmole TE/ |
| | | milliliter |
| Total ORAC5.0 (sum of above) | 105.22** | μmole |
| | Equivalent to | TE/milliliter |
| | 340/gram | |

Kelp Oil and Krill Oil Mix, Oil

### Results:

| Test | Result | Units |
|---|---|---|
| Antioxidant power against peroxyl radicals | 173 | μmole |
| | | TE/gram |
| Antioxidant power against hydroxyl radicals | 181 | μmole |
| | | TE/gram |
| Antioxidant power against peroxynitrite | Not Detected | μmole |
| | | TE/gram |
| Antioxidant power against super oxide anion | Not Detected | μmole |
| | | TE/gram |
| Antioxidant power against singlet oxygen | 63 | μmole |
| | | TE/gram |
| Total ORAC_{FN} (sum of above) | 417 | μmole |
| | | TE/gram |

87% Fucoidan and Krill Oil Mix, Oil

### Results:

| Test | Result | Units |
|---|---|---|
| Antioxidant power against peroxyl radicals | 123 | μmole |
| | | TE/gram |
| Antioxidant power against hydroxyl radicals | 206 | μmole |
| | | TE/gram |
| Antioxidant power against peroxynitrite | Not Detected | μmole |
| | | TE/gram |
| Antioxidant power against super oxide anion | Not Detected | μmole |
| | | TE/gram |
| Antioxidant power against singlet oxygen | Not Detected | μmole |
| | | TE/gram |
| Total ORAC_{FN} (sum of above) | 329 | μmole |
| | | TE/gram |

Kelp Concentrate

### Results:

| Test | Result | Units |
|---|---|---|
| Antioxidant power against peroxyl radicals | 195 | μmole |
| | | TE/gram |
| Antioxidant power against hydroxyl radicals | 828 | μmole |
| | | TE/gram |
| Antioxidant power against peroxynitrite | 6 | μmole |
| | | TE/gram |
| Antioxidant power against super oxide anion | 64 | μmole |
| | | TE/gram |
| Antioxidant power against singlet oxygen | 652 | μmole |
| | | TE/gram |
| Total ORAC_{FN} (sum of above) | 1,745 | μmole |
| | | TE/gram |

90.2% Fucoidan and Krill Oil, Thick Liquid

### Results:

| Test | Result | Units |
|---|---|---|
| Antioxidant power against peroxyl radicals | 818 | μmole |
| | | TE/gram |
| Antioxidant power against hydroxyl radicals | 190 | μmole |
| | | TE/gram |
| Antioxidant power against peroxynitrite | 9 | μmole |
| | | TE/gram |
| Antioxidant power against super oxide anion | 0 | μmole |
| | | TE/gram |
| Antioxidant power against singlet oxygen | 56 | μmole |
| | | TE/gram |
| Total ORAC_{FN} (sum of above) | 1,073 | μmole |
| | | TE/gram |

Total Phenolics

### Results:

| Description | BL ID | Test | Result | Units |
|---|---|---|---|---|
| 87% Fucoidan Extract of | 10-2235 | Total Phenolics | 6.54 | mg/gram |
| Macrocystis Pyrifera, | | | | |
| Powder | | | | |

The phenolic result is expressed as milligram gallic acid equivalency.

### Results:

| Description | BL ID | Test | Result | Units |
|---|---|---|---|---|
| Liquid Kelp Oil, | 10-2297 | Total Phenolics | 0.74 | mg/mililiter |
| Liquid, Lot #1 | | | | |

The phenolic result is expressed as milligram gallic acid equivalency.

Scavenging Capacity

### Scavenging Capacity of Krill Oil and Liquid Sieve Sap on UVA/UVB Induced Free Radicals

Conc. (μg/ml)

| | | Liquid Sieve | Scavenging |
|---|---|---|---|
| Treatment | Krill Oil | Sap | Capacity Rate (%) |
| Sample | 1066.24 | 266.56 | 81.44 |
| extracts + | 533.12 | 133.28 | 72.45 |
| UVB/UVA | 266.56 | 66.64 | 51.29 |
| | 133.28 | 33.32 | 45.76 |
| | 66.64 | 16.66 | 47.87 |
| | 33.32 | 8.33 | 38.62 |
| | 16.66 | 4.17 | 31.68 |
| | 8.33 | 2.08 | 21.94 |

### Scavenging Capacity of Fucoidan and Krill Oil on UVA/UVB Induced Free Radicals

| | |
|---|---|
| Conc. (μg/ml) | Scavenging |

| Treatment | Fucoidan | Krill oil | Capacity Rate (%) |
|---|---|---|---|
| Sample | 666.4 | 666.4 | 88.97% |
| extracts + | 333.2 | 333.2 | 80.00% |
| UVB/UVA | 166.6 | 166.6 | 66.78% |
| | 83.3 | 83.3 | 58.61% |
| | 41.65 | 41.65 | 41.62% |
| | 20.83 | 20.83 | 24.62% |
| | 10.41 | 10.41 | 9.68% |
| | 5.21 | 5.21 | 0.75% |

D. Discussion of ORAC Results and their Significance

ORAC 5.0 Values of Macrocystis pyrifera

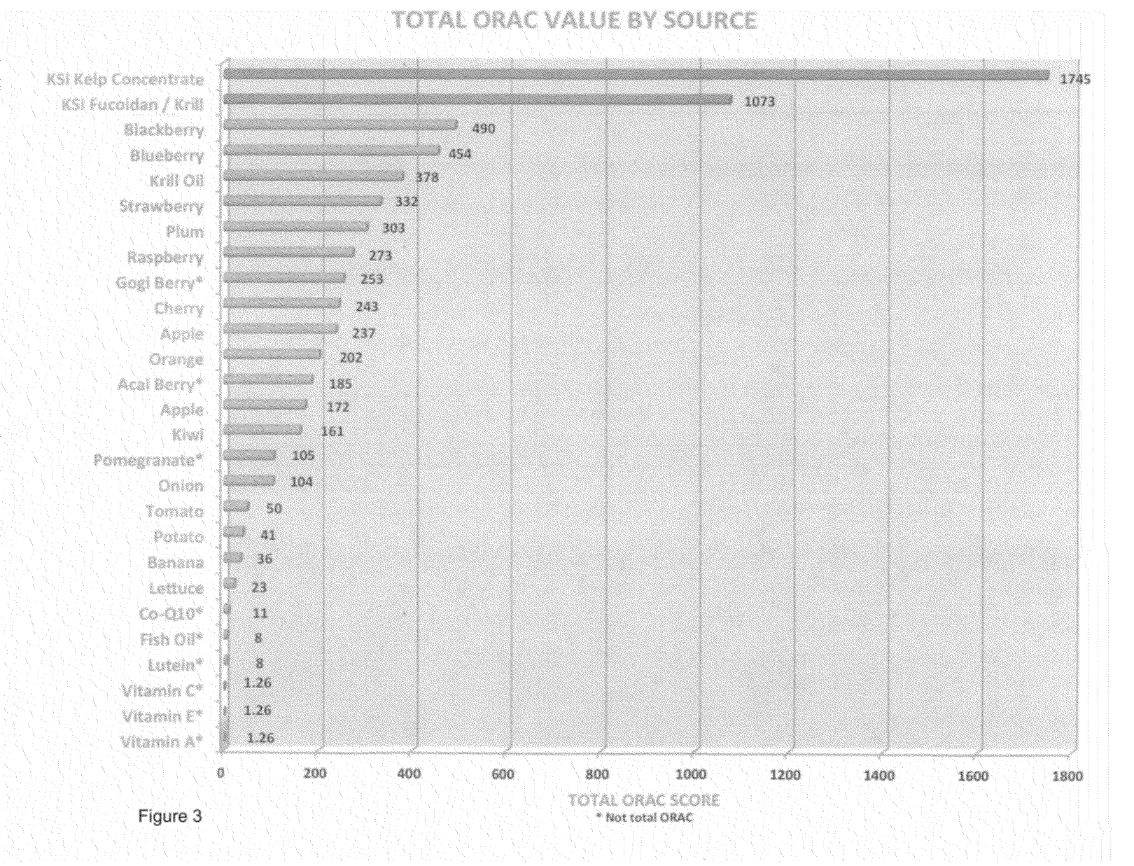

The ORAC5.0 values from testing of Macrocystis pyrifera derived High-Purity Fucoidan and the Kelp Oil and Kelp Concentrate of Macrocystis pyrifera were truly unexpected and surprising results. In fact, as shown in FIG. 3, Kelp Concentrate had a Total ORAC5.0 value 1,745/umole/gram. This is greater than any natural food product ever tested including all Vitamins, fish oil, and the leading marketed AntiOxidant sources (namely pomegranate, cranberries, blue berries, gogi berry, and acai berry) as graphically shown in FIG. 3. In addition, when Kelp Oil, with an ORAC of 340/gram was combined with Krill Oil, with an ORAC of 378/umole/gram, the Total ORAC5.0 value had a synergistic impact boosting the overall ORAC total value to 417 per gram. When 90.2% High Purity Fucoidan, with a Total ORAC5.0 of 131/umole/gram, was combined with Krill Oil (50/50) the Total ORAC5.0 was 1,073/umole/gram. This is a remarkable result. The inventors believe the escalating purity of Fucoidan, even with a lower ORAC5.0, triggers a sharp bioactive boost in the joint ORAC5.0 of the blend with Krill Oil, itself only at 378/umole/gram. What is also revealing is that the tested “scavenging capacity” of these combinations were up to 80% or greater, indicated their efficiency in destroying the five free radicals on consumption. Total phenolics for Fucoidan were greater than for Kelp Oil. Phenolics consisting of the polyphenol compound is one marker for the Total ORAC scores expected to occur in the compound. However the inventors found that the Higher Purity Fucoidan with its higher phenolics would also impact positively the joint ORAC 5.0 score. In other words, the scores were not just additive. One combination impact to date has focused on combining their compounds with Krill Oil which has an ORAC of 378.

When High Purity (78%) Kelp Oil was combined with Krill Oil, at 50/50, the Total ORAC5.0 value had a synergistic impact boosting the overall ORAC total value to 412/umole/gram. But with the new Higher Purity (90.2%) Fucoidan with Krill Oil, the Total ORAC5.0 score was 1,073/umole/gram, a significant material improvement with higher quality Fucoidan even with a less ORAC5.0 value of 131/umole/gram. The present inventors believe that with increasing purity of Fucoidan, which means lesser amounts of indigenous Kelp Concentrate in the compound as extract purity is increased, the bioactive result is even higher Total combined ORAC, and hence a greater AntiOxidant impact. As noted above the tested “scavenging capacity” of our compound combinations were up to 80% or greater, indicating their efficiency in counteracting the five free radicals and implying a reduction in oxidative stress after consumption. Total phenolics for 78% pure Fucoidan were greater than Kelp Oil, at 6.75. In the tables above reference to “sieve sap” is Kelp Oil.

The Kelp Oil acquired from Macrocystis pyrifera off the coast of California was further processed to reduce the moisture to create Kelp Concentrate. Moisture content is a key starting point for accumulation of Kelp Oil. Macrocystis pyrifera has an 84% moisture content and it is presently believed that this high moisture level combined with the size of the Macrocystis plant (versus all other brown alga) directly contributes to the volume and characteristics of the Kelp Concentrate. In turn the present inventors believe that drying the Kelp Oil yields the high AntiOxidant capacity and that this can apply to all brown macro alga with a moisture content greater than 50%. The Kelp Concentrate residue suitable for use with various aspects of the present invention are derived from the Kelp Oil of any suitable brown algae (with the highest yielding Kelp Oil supply that could support a commercial operation coming from Macrocystis pyrifera), including all of the Macrocystis species, Macrocystis pyrifera, Macrocystis integrifolia, and angustifolia; the 28 species of Laminaria, including Laminaria japonica, Laminaria pinnatifida, and Laminaria hyperborean; the nine species of Ecklonia, Ascophyllum nodosum; the 13 species of Fucus; the 350 species of Sargassum; and the 22 species of Turbinaria and all other brown macro algaes.

The TOTAL ORAC5.0 scores of Kelp Concentrate surpassed all expectations and topped the scores of all previous testing. Total ORAC5.0 for Kelp Concentrate was 1,745 per/umole/gram. As will be appreciated, there are thousands of products claiming “AntiOxidants” that the inventors believe if blended with Kelp Concentrate could see a material boost in actual AntiOxidant power.

The “boost” is clear given that the AntiOxidant power or TOTAL ORAC5.0 of Kelp Oil (340/umole/gram) is increased by over five times by conversion to Kelp Concentrate (1,745/umole/gram). It is also noted that High Purity Fucoidan in combination with Krill Oil had a TOTAL ORAC5.0 score of 1,073/umole/gram, again verifying the unexpected synergy that High Purity Fucoidan, Kelp Concentrate and Krill Oil have that can be used in multiple hybrid health, wellness, and food products adding Omega 3 content and higher overall AntiOxidant power. The inventors see that in most cases in Functional Food products, when marketing labels on, for example, breakfast cereals and similar products claim “AntiOxidants” they are largely referring to Vitamin ingredients (A, B, C, D, E) in portions so minor as not to provide any true AntiOxidant benefit at all. Furthermore, even at 100% MDR, most vitamins have poor starting AntiOxidant power in terms of Total ORAC5.0. The inventors believe their compounds will materially boost the true AntiOxidant power of these products per serving. The inventors also believe their compounds will provide the desired anti-aging/anti-oxidative stress benefits to the thousands of cosmeceutical products now claiming to include beneficial “antioxidants” which typically are Vitamins E and A in small portions and alternative popular berry extracts and even spices such a curcumen (turmeric).

These ORAC5.0 results also indicate that High Purity Fucoidan and Kelp Concentrate may have specialized new applications in the medical field, as for example, an efficient carrier of AntiOxidant power in currently manufactured bone void fillers and cements for orthopedic applications, both made with calcium and calcium phosphates or with magnesium oxide. These orthopedic bone void fillers and cements that are also resorbable, biocompatible, and osteoconductive can provide for a reduction of oxidative stress in the patient surgical site and potentially reinforce the “binder” features of some of these products. Another potential medical use conceived by the inventors is the use of a radioactive labeled High Purity Fucoidan to identify clots, specifically atrial clots and pulmonary emboli. As will be appreciated, currently atrial clots are only positively identified with a transesolphageal echocardiogram which requires anesthesia to perform on patients who have atrial fibrillation. These tests are then used to determine if a patient can be cardioverted or needs Coumadin long term. With the present invention, a SPECT imaging scan, or other nuclear imaging modality may be used to search for a pulmonary embolism. Using a radioactive tracer with High Purity Fucoidan or Kelp Concentrate to search for clots would be a major breakthrough in treating patients with these conditions.

According to the invention, High Purity Fucoidan and Kelp Concentrate could also be used to combat the effects of aging by offsetting the special category of cells known as “senescent cells”, which promote the aging on the tissues. By helping to cleanse the body of these cells, you postpone many of the diseases of aging. Given the starting point that Kelp Concentrate and Fucoidan both have high scores to apply to the skin damaging two free radicals: hydroxyl radicals (HORAC) and singlet oxygen (SOAC), To be more specific, Senescent cells accumulate in aging tissues, like arthritic knees, cataracts and the plaque that may line elderly arteries. The cells secrete agents that stimulate the immune system and cause low-level inflammation. It has recently been concluded that the presence of these senescent cells are a negative and the present inventors believe that High Purity Fucoidan and Kelp Concentrate may have a positive role in offsetting the negative impact of these cells, much like it does with the Five Free Radicals, in particular for skin, hydroxyl radicals and singlet oxygen.

D. Free Radical Cell Viability and Cellular Antioxidant Activities Testing

Two test assays were completed with the 78% Fucoidan and Kelp Oil to determine cell penetration and protection capabilities of these materials when applied to human cells. The results discussed below showed these products had the ability to give added protection to skin cells from anti-aging effects (wrinkled skin and brown spots) and affirmed the positive 78% Fucoidan ORAC5.0 test results for the two key Free Radicals that relate to skin oxidative stress. AAPH is a chemical that generates free radicals. It is one of the most commonly used radical generators used in assays. It is like adding yeast to trigger fermentation. The cellular antioxidant assays were positive for Fucoidan. They demonstrate that, at concentrations that have limited to no toxicity to the cells, the (78%) Fucoidan and Kelp Oil/sieve sap samples inhibit oxidation induced by the radical generator AAPH by 21.16% and 65.77%, respectively. The in vitro IC50 assays indicate that the Fucoidan and Kelp Oil/sieve sap samples do not inhibit elastase or collagenase, enzymes that damage elastin and collagen proteins. IC50 is a standard methodology for measuring inhibition. It stands for the concentrations of a substance at which a specified reaction is inhibited by 50%. The inventors plan to do additional testing with the 90.2% Fucoidan and the Kelp Concentrate (as opposed to Kelp Oil/sieve sap) to measure those results.

Sample Preparation

Accurately weight 10 mg of powder sample (78% Fucoidan Extract of Macrocystis pyrifera) to a 1.5 mL tube. Add 1200 μl solution (DMSO: PBS=80:20).

2. Cell Culture

T24 cells were cultured in DMEM/F12 medium with 10% fetal bovine serum, L-glutamine, and penicillin-streptomycin-neomycin [1]. Cells were seeded (1×105/ml) to a 96 well plate and cultured overnight.

3. Cell Viability Assay for Two Samples

Cells were cultured overnight. Then serially diluted samples were added into each well. The bioluminescent ATP assay kit was used to evaluate the cell viability.

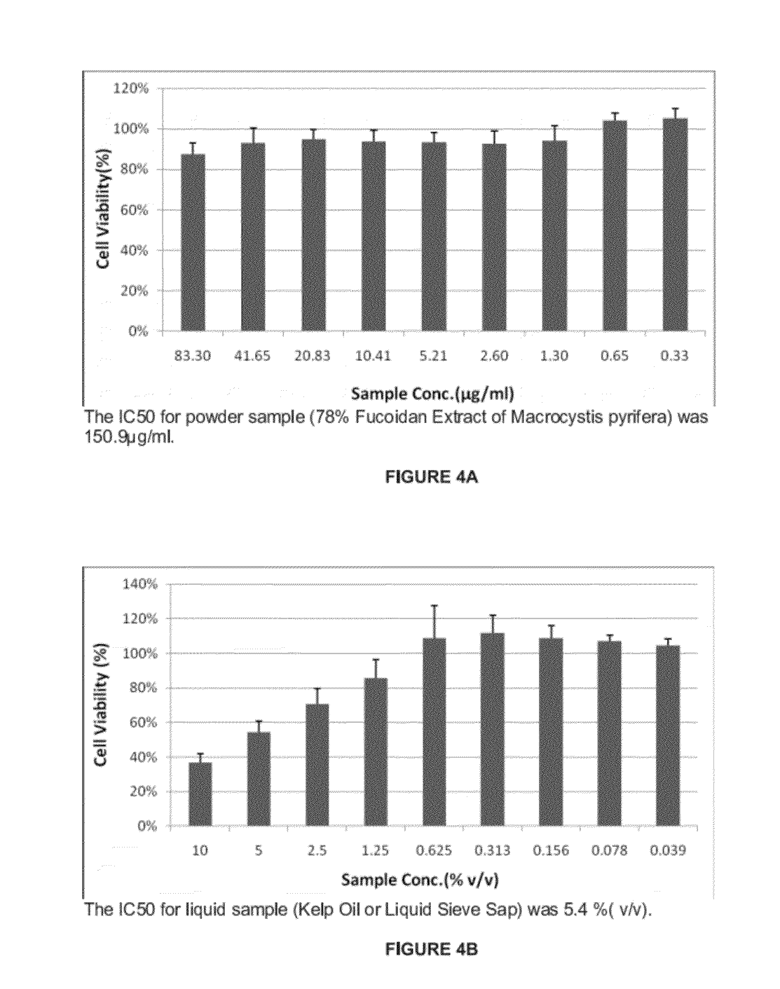

Results: Shown in FIG. 4A is the IC50 of 78% Fucoidan. Shown in FIG. 4B is the IC50 of Kelp Oil.

4. Cellular Antioxidant Activity (CAA) Assay

Cells were cultured overnight. Then the culture medium was removed and the cells were washed with PBS twice. Cells were treated with 200 μl of 50 μmol/L DCFU-DA (2′,7′-Dichlorofluorescin diacetate) dissolved in culture medium or culture medium for 30 minutes at 37° C. with 5% CO2. Cells were washed with PBS twice after incubation. 20 mM AAPH (2,2′-azobis-2-methyl-propanimidamide, dihydrochloride), serial diluted samples (based on cell viability result), resveratrol and culture medium were add into each well and the 96-well plate was placed into a fluorescence plate-reader at 37° C. Emission at 538 nm was measured with excitation at 485 nm every 5 minutes for 2 hours.

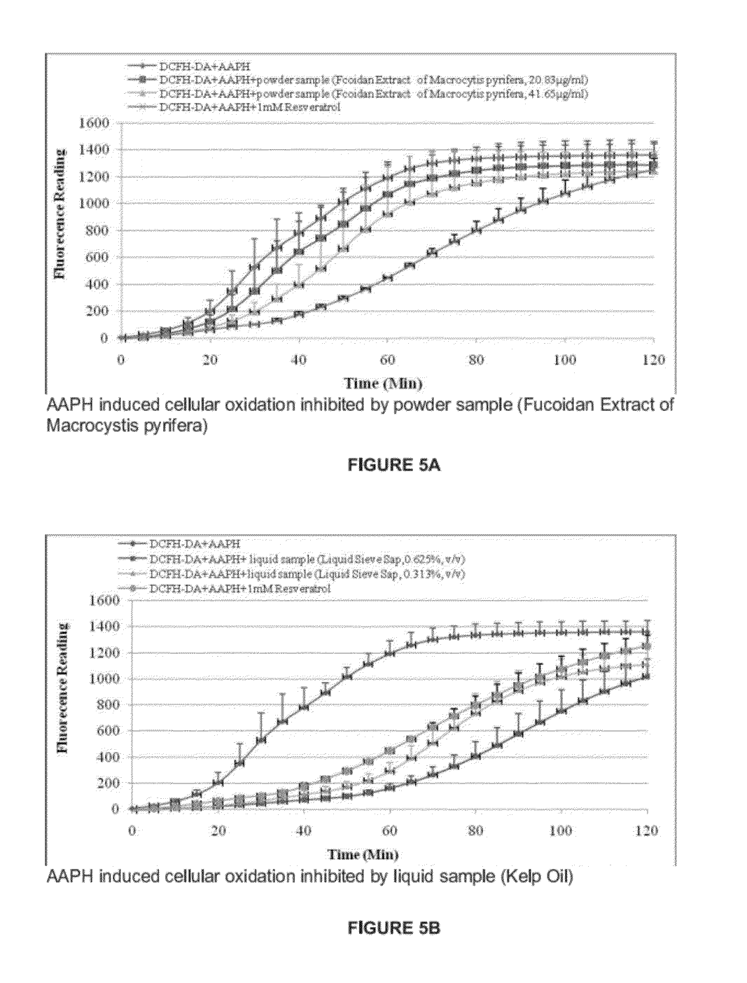

Results: Shown in FIG. 5A is the AAHP CAA assay results for 78% Fucoidan. Shown in FIG. 5B the AAHP CAA assay results for Kelp Oil.

E. Discussion of the Antioxidant Benefits of Macrocystis Pyrifera Derived Products

As shown in FIG. 3, Kelp Concentrate had an ORAC value greater than any natural food product ever tested as well as all vitamins, fish oils, and other known nutritional AntiOxidant sources (namely pomegranate, cranberries, blue berries, gogi berry, and acai berry. In operation, a potential utility for this invention is providing the Functional Foods, Cosmeceuticals/Nutricosmetics, and Dietary Supplement industries a safe, organic AntiOxidant compound to use in products directed to offsetting the negative impacts of oxidative stress from the Five Free Radicals. Again, not wishing to be bound by theory but the present invention shows a synergistic effect on AnitOxidant power when Macrocystis pyrifera derived materials are added to existing AntiOxidant sources. For example, when Kelp Oil was combined with a popular ingredient for Omega-3 content, Krill Oil, the ORAC score was 417 umole/gram while Krill Oil itself only has an ORAC score of 378 umole/gram according to the product's GRAS filings and Kelp Oil only has an ORAC score of 340/umole/gram. Testing also revealed that while 78% Fucoidan tested at 314/umole/gram, when combined with Krill Oil the ORAC score fell to 329/umole/gram. This phenomena suggests that the higher the purity of Fucoidan, which in turn removes more of the remaining Kelp Concentrate to get to that purity, the lower the ORAC value. However, additional testing showed that a blend of higher Fucoidan purity products with other compounds have the best benefit as they demonstrate bioactive results in terms of ORAC value. For example, when 90.2% Fucoidan was combined with Krill Oil, there were dynamic effects in that the Total ORAC score jumped to 1,073/umole/gram from the 340/umole/gram results when 78% Fucoidan was used (Kelp Concentrate alone is 1,745/umole/gram).

IV Macrocystis Pyrifera and its Surprising and Unexpectedly Superior Anti-Viral Activity

The anti-viral activity of Macrocystis pyrifera derived High-purity Fucoidan demonstrated in the in vitro test results set forth below suggests that the present inventors may have achieved the long-felt, yet unresolved need in the art for finding a cure to the world's HIV/AIDS epidemic as well as providing an economically feasible prevention, treatment, and cure for many thought un-curable and oft untreatable viruses.

A. Antiviral Assay Tests

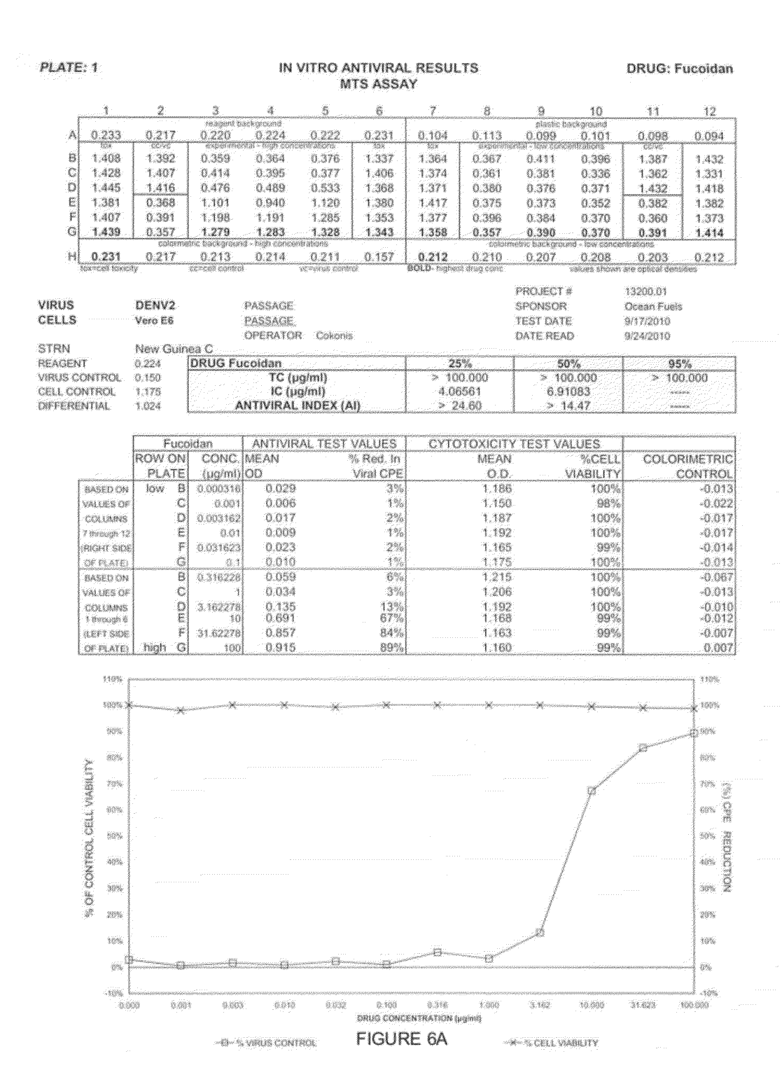

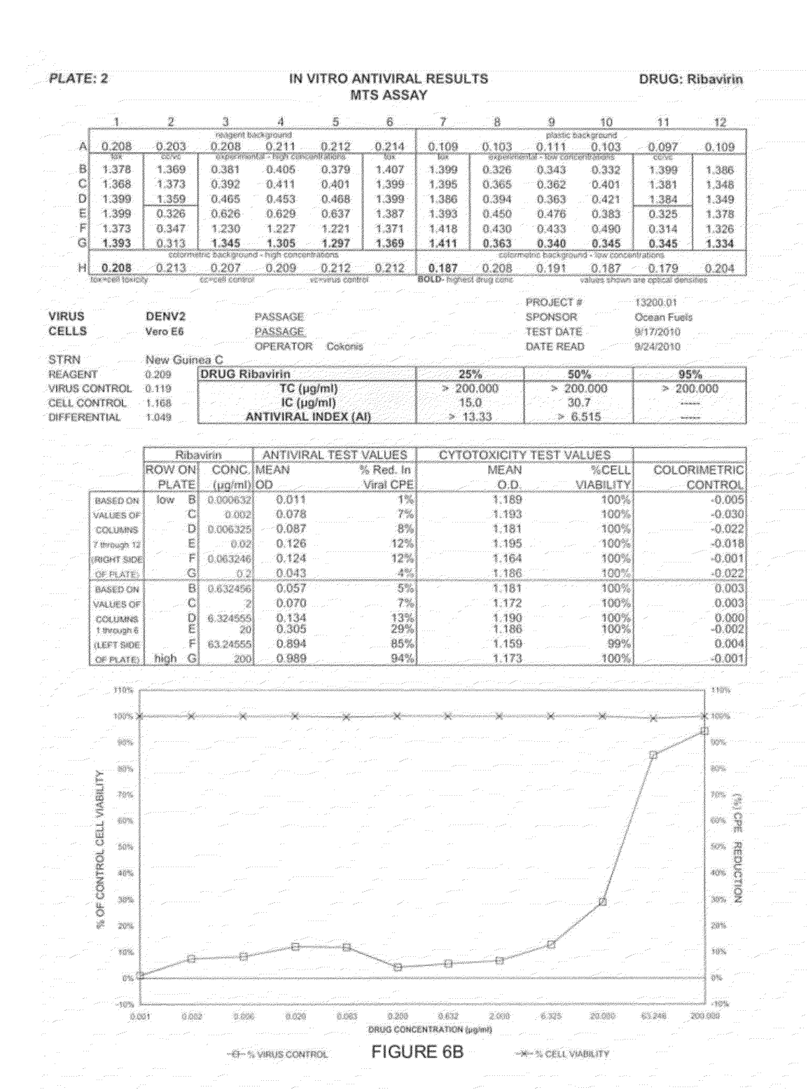

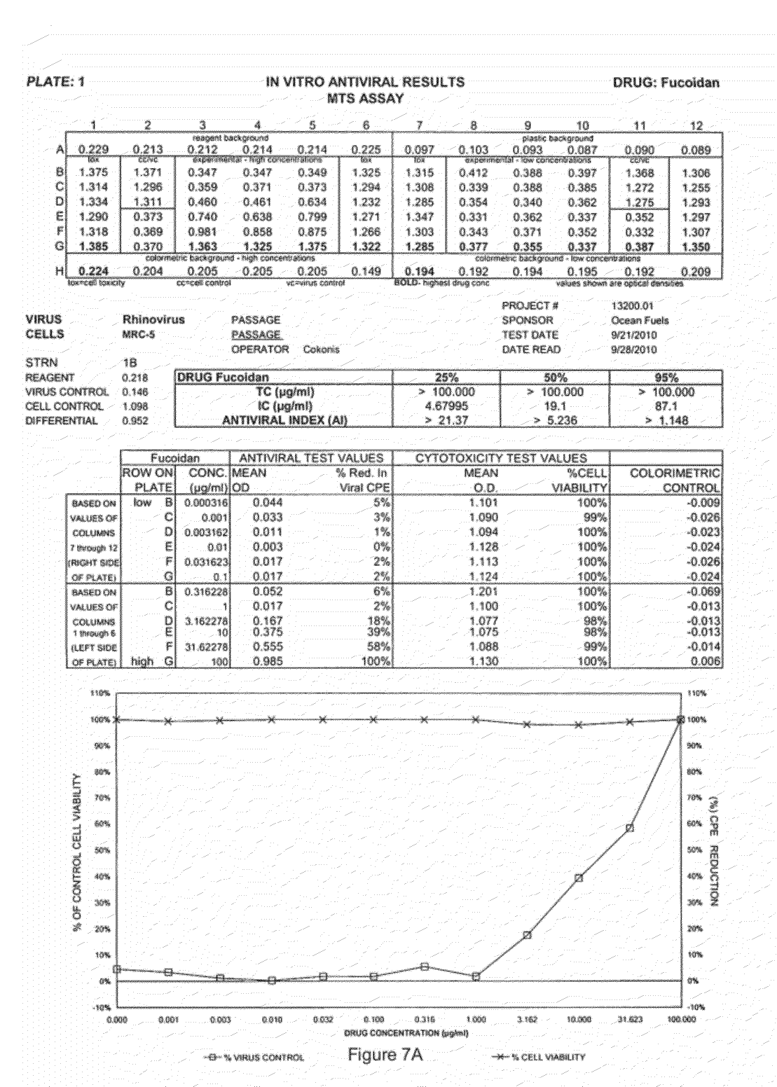

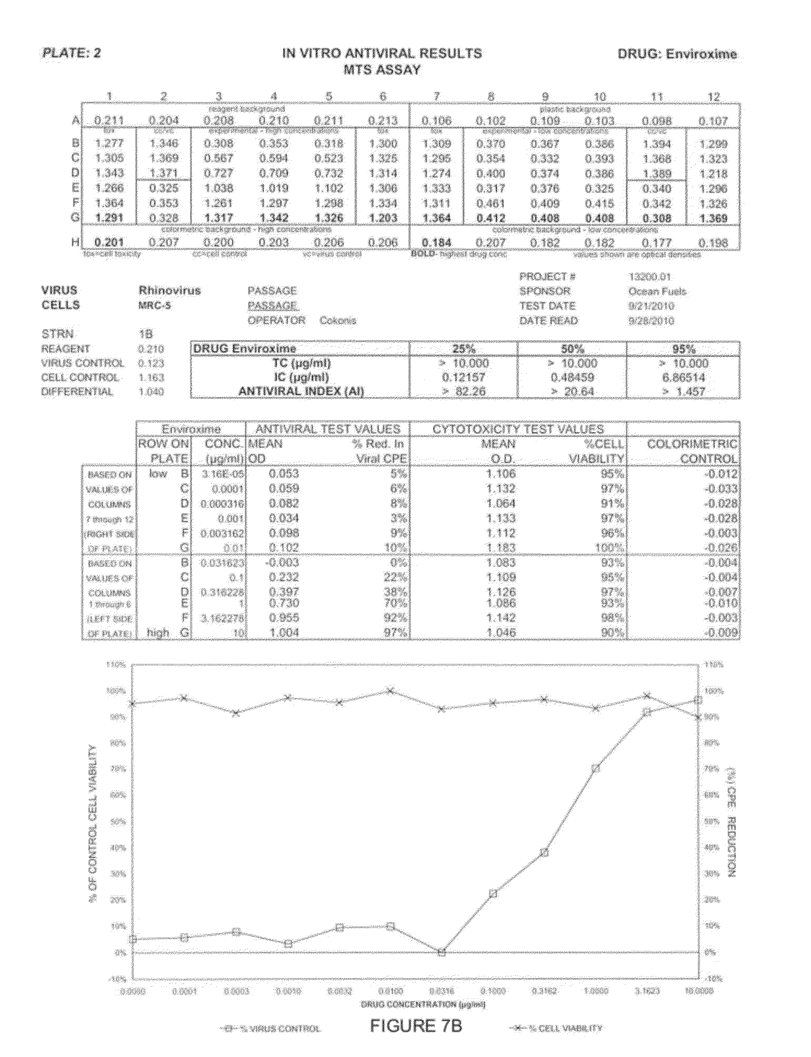

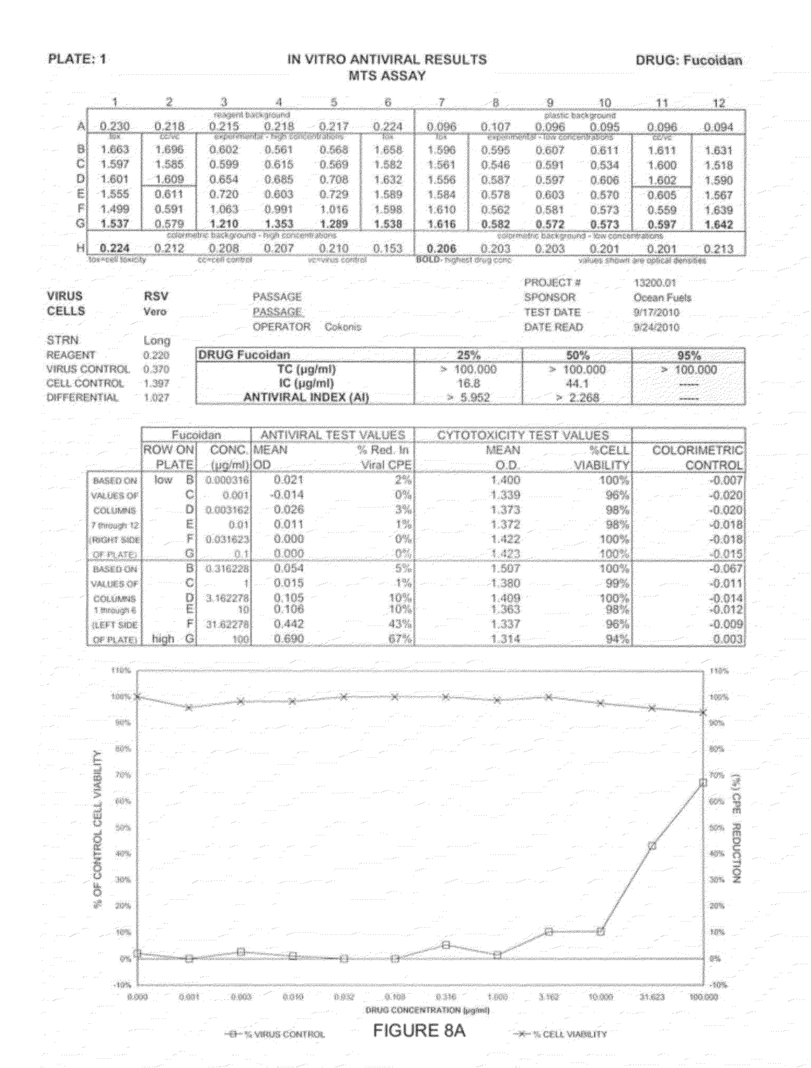

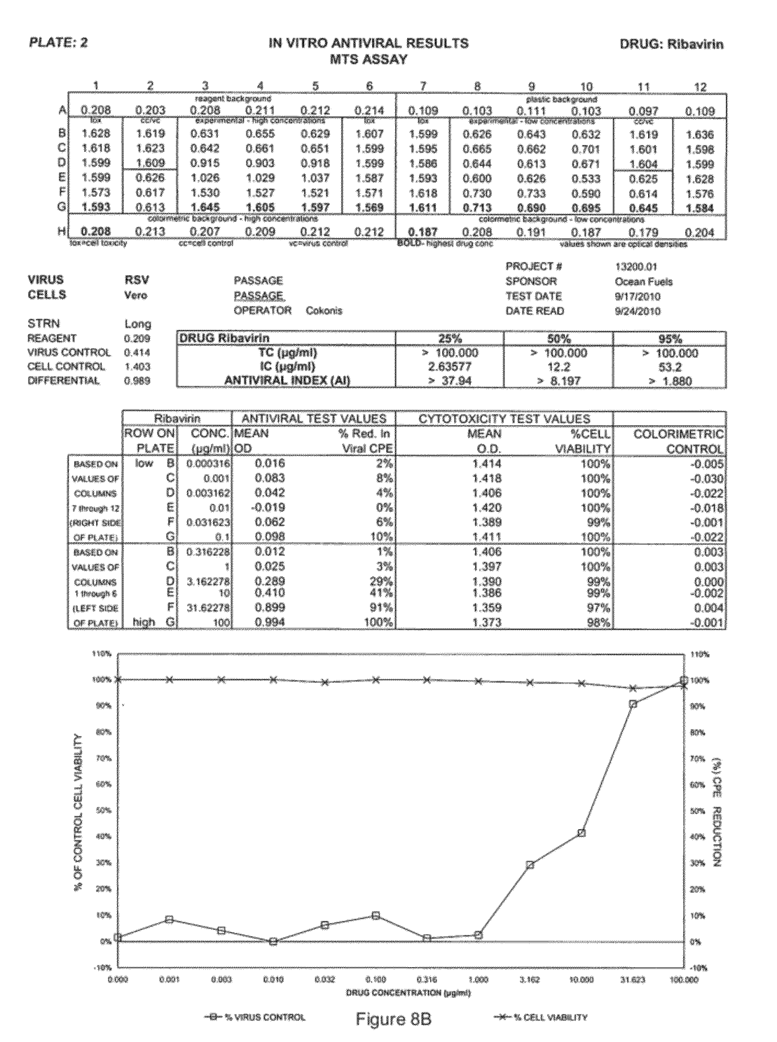

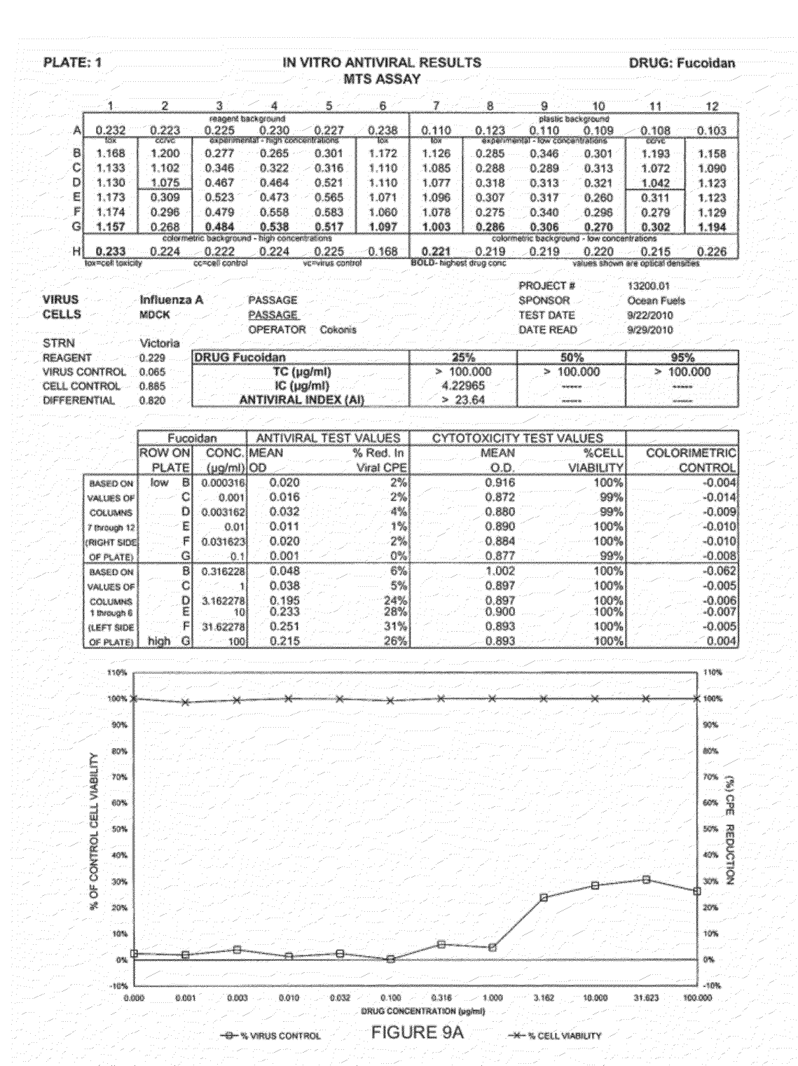

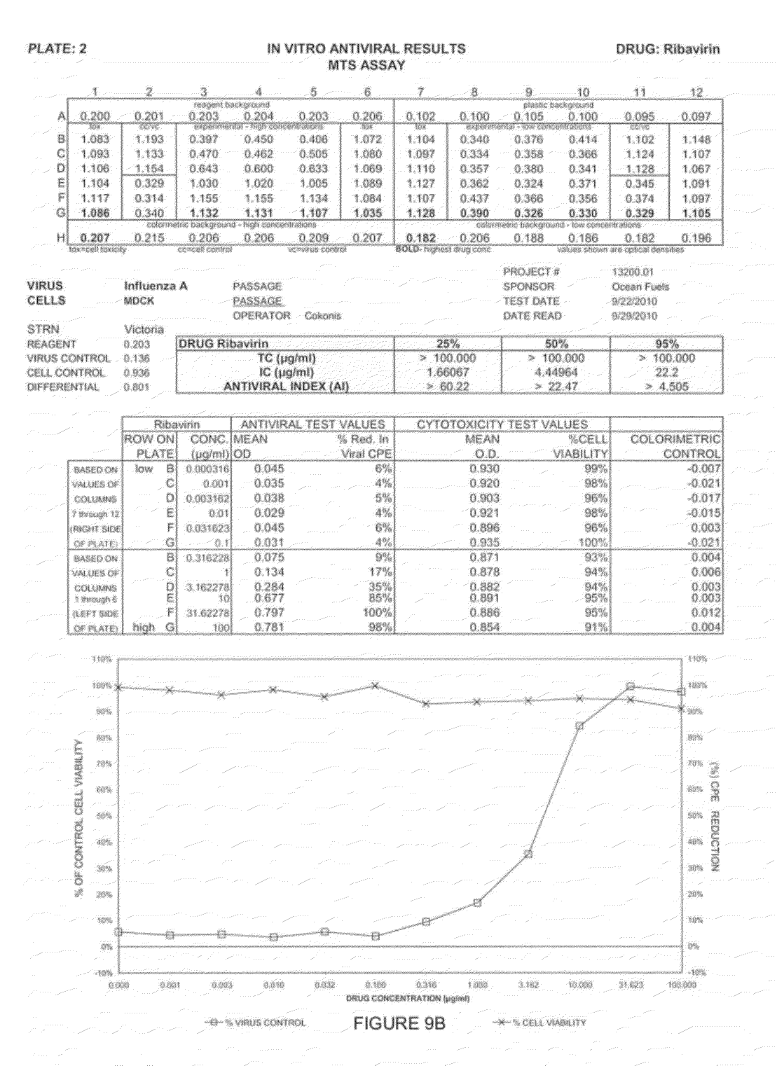

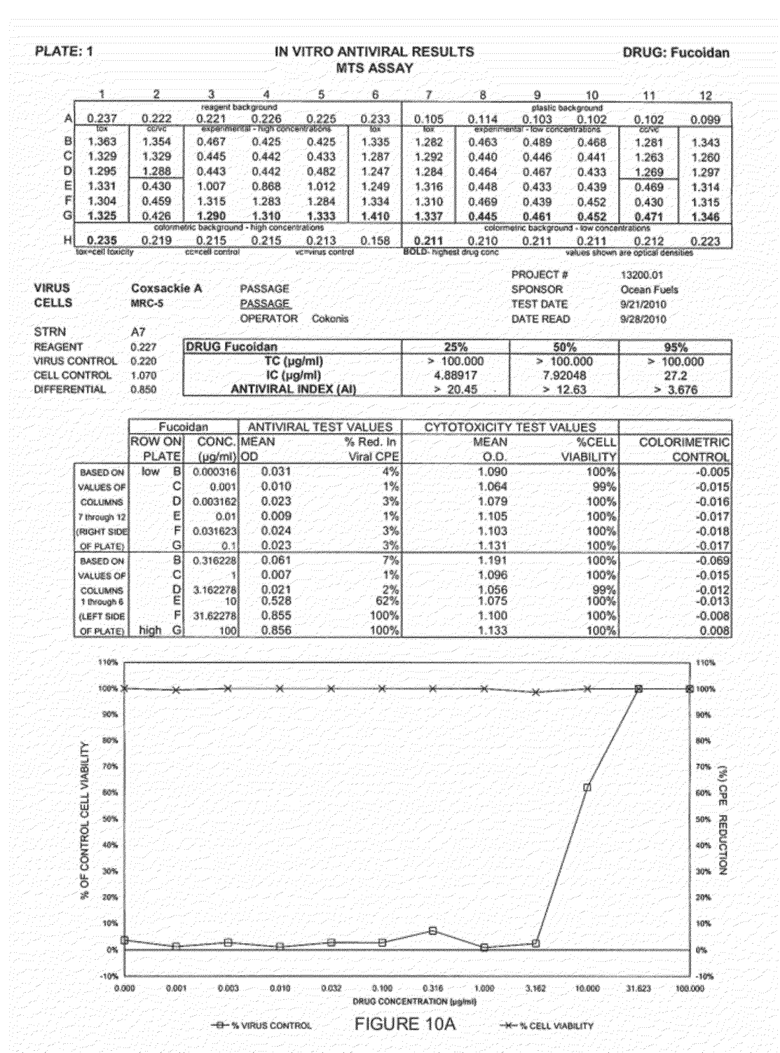

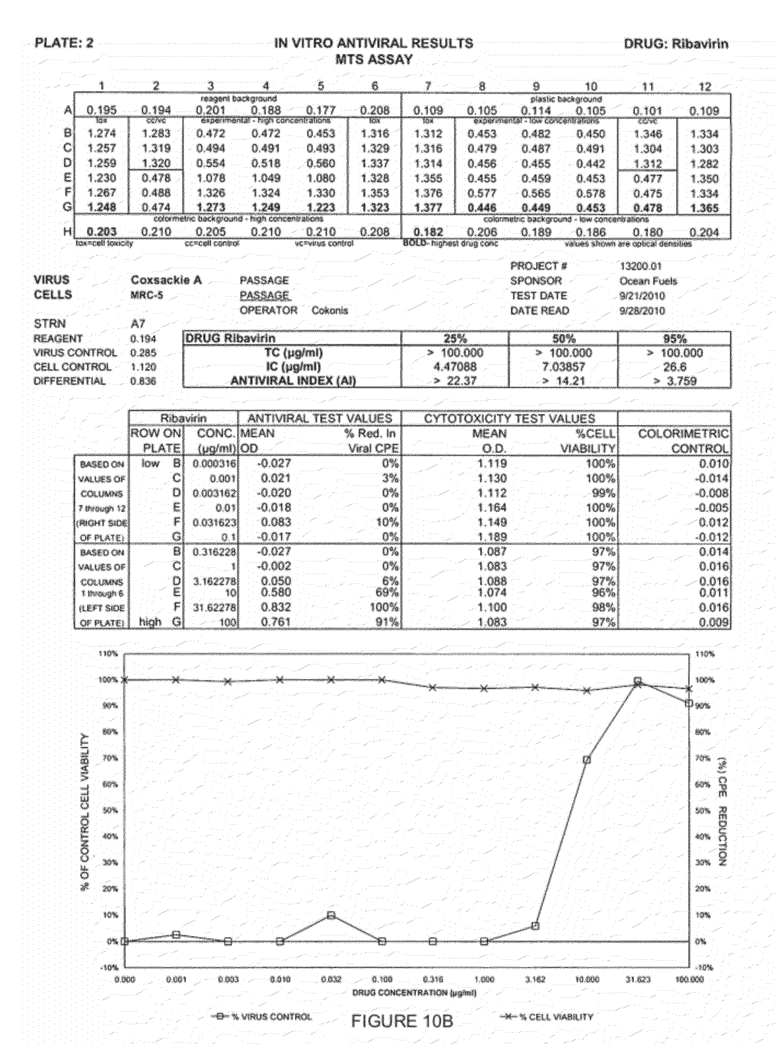

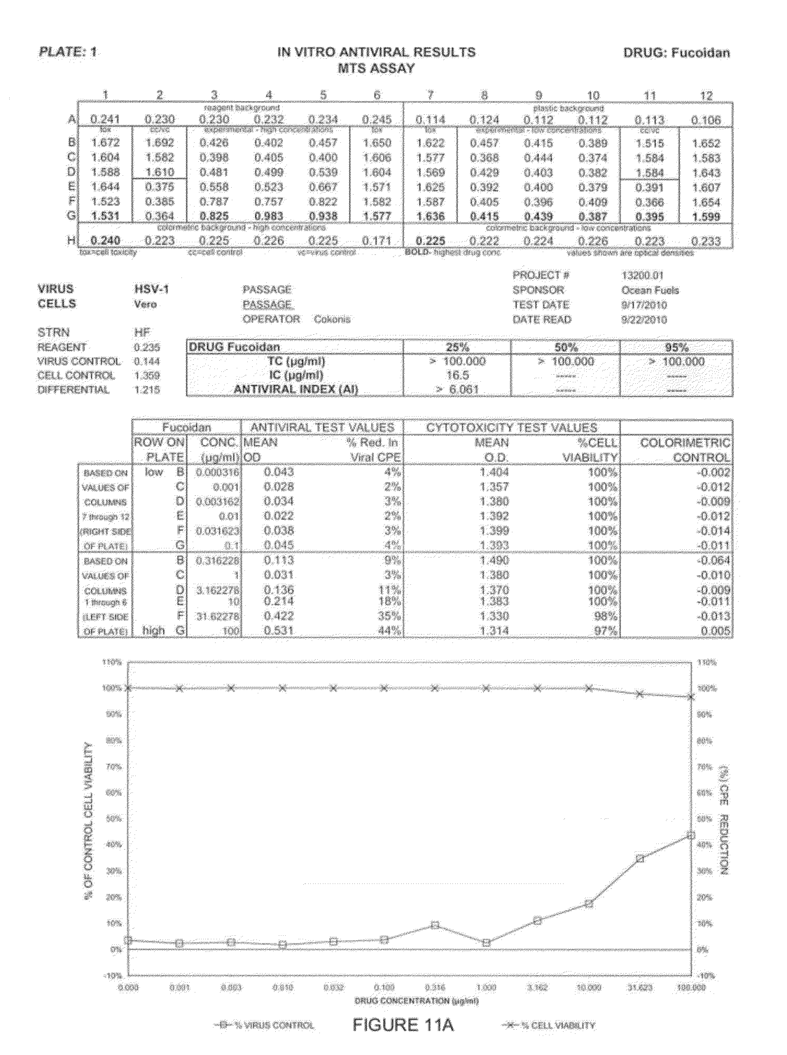

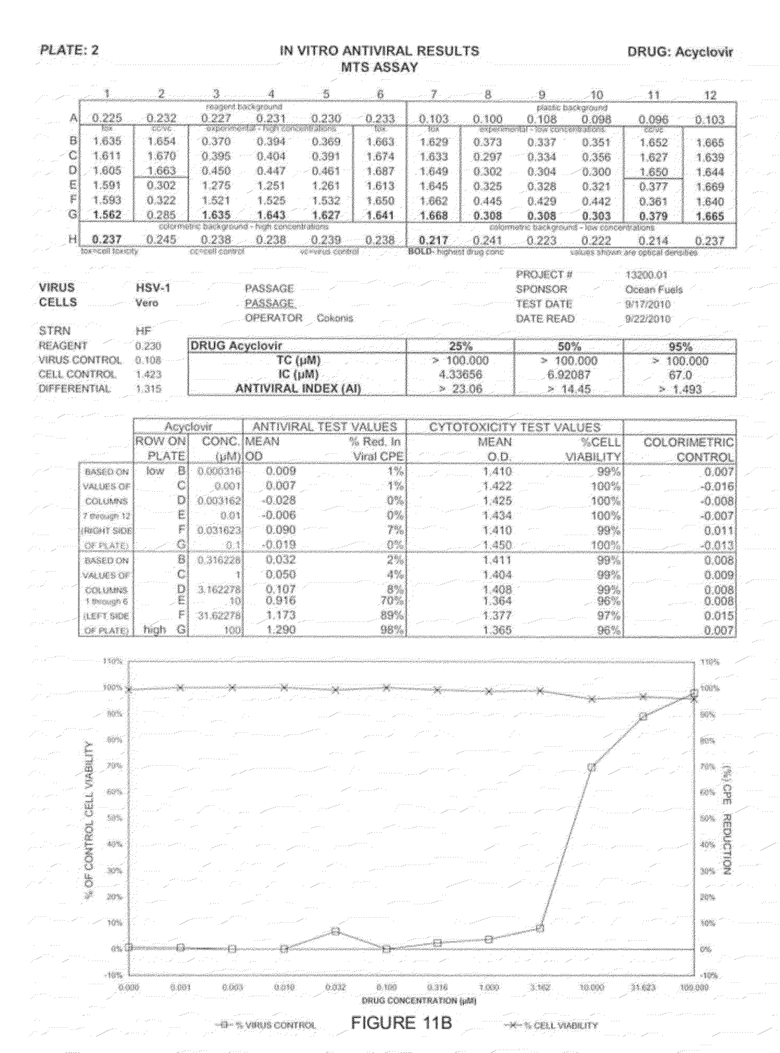

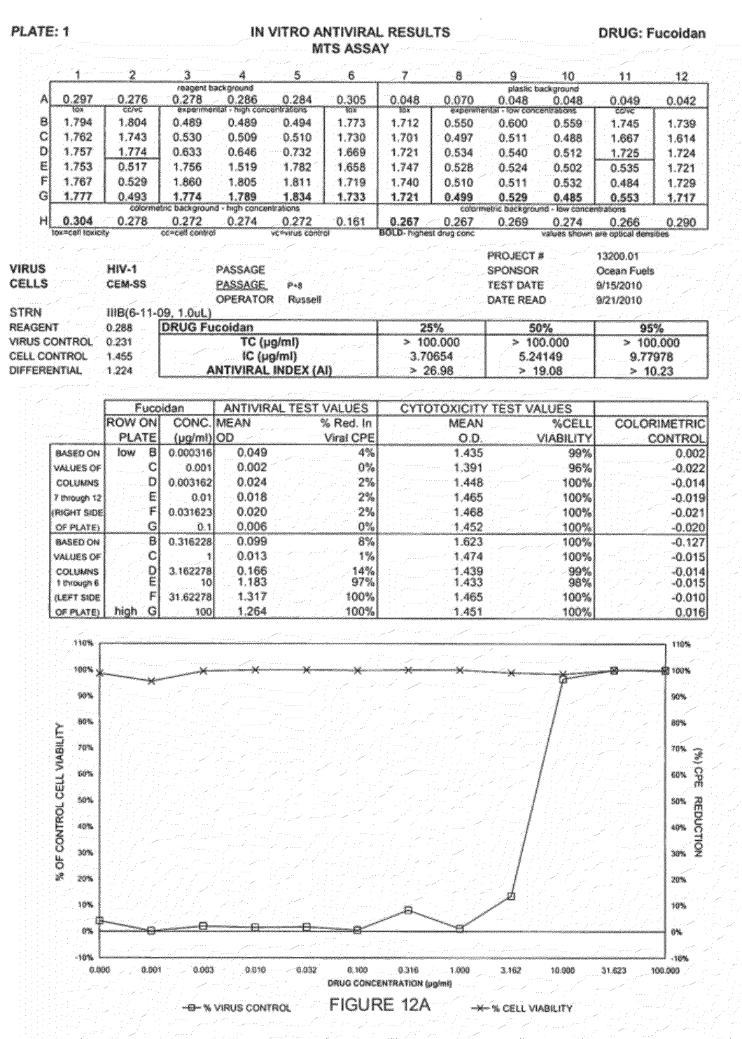

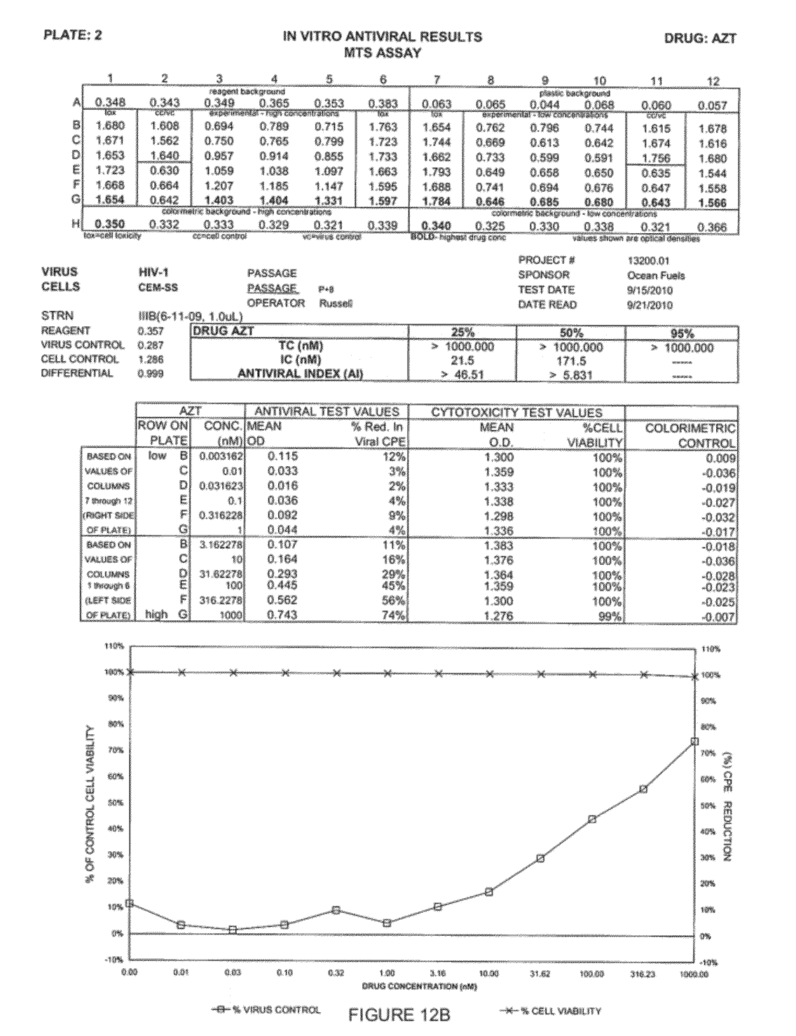

The specifics of each assay are presented individually in more detail below. The assay test results are presented individually in respective FIGS. 6A,B-12A,B.

B. Description of the Dengue Virus Cytoprotection Assay

The Dengue Virus Cytoprotection assay uses Vero E6 cells and Dengue Virus Type 2 strain New Guinea C. Briefly virus and cells are mixed in the presence of test compound and incubated for 7 days. The virus is pre-titered such that control wells exhibit 85 to 95% loss of cell viability due to virus replication. Therefore, antiviral effect or cytoprotection is observed when compounds prevent virus replication. Each assay plate contains cell control wells (cells only), virus control wells (cells plus virus), compound toxicity control wells (cells plus compound only), compound colorimetric control wells (compound only), as well as experimental wells (compound plus cells plus virus). Cytoprotection and compound cytotoxicity are assessed by MTS (CellTiter®96 Reagent, Promega, Madison Wis.) dye reduction. The % reduction in viral cytopathic effects (CPE) is determined and reported; IC₅₀ (concentration inhibiting virus replication by 50%), TC₅₀ (concentration resulting in 50% cell death) and a calculated TI (therapeutic index=TC₅₀/IC₅₀) are provided along with a graphical representation of the antiviral activity and compound cytotoxicity when compounds are tested in dose-response. Each assay includes ribavirin as a positive control.

Cell Preparation

Vero E6 cells (Kidney, African green monkey, Cercopithecus aethiops, clone) were obtained from the American Type Culture Collection (ATCC, Rockville, Md.) and are grown in Eagle's Minimum Essential Medium with Earle's BSS (EMEM) supplemented with 10% fetal bovine serum (FBS), 2.0 mM L-Glutamine, 100 units/ml Penicillin and 100 ug/ml Streptomycin. Cells are sub-cultured twice a week at a split ratio of 1:4 using standard cell culture techniques. Total cell number and percent viability determinations are performed using a hemacytometer and trypan blue exclusion. Cell viability must be greater than 95% for the cells to be utilized in the assay. The cells are seeded in 96-well tissue culture plates the day before the assay at a concentration of 5×10³ cells/well.

Virus Preparation

The virus used for this assay is Dengue Virus Type 2 strain New Guinea C. This virus was obtained from the American Type Culture Collection (ATCC) and was grown in Vero E6 cells for the production of stock virus pools. For each assay, a pre-titered aliquot of virus is removed from the freezer (−80° C.) and allowed to thaw slowly to room temperature in a biological safety cabinet. The virus is re-suspended and diluted into tissue culture medium such that the amount of virus added to each well is the amount determined to give between 85 to 95% cell killing at 6-7 days post-infection.

Plate Format

A standardized plate format is used for cytoprotection assays. Each plate contains cell control wells (cells only), virus control wells (cells plus virus), drug cytotoxicity wells (cells plus drug only), drug colorimetric control wells (drug only), background control wells (media only), as well as experimental wells (drug plus cells plus virus). Samples are evaluated for antiviral efficacy with triplicate measurements using 6 concentrations at half-log dilutions (12 concentrations can also be performed) in order to determine IC₅₀ values and with duplicate measurements to determine cytotoxicity, if detectable. The table below represents the standard plate format for testing compounds at 6 concentrations using a representative high-test concentration of 100 μM.

TABLE
Plate Format for Dengue Virus Cytoprotection Assays

| | 1 | 2 | 3 | 4 | 5 | 6 | 7 | 8 | 9 | 10 | 11 | 12 |
|---|---|---|---|---|---|---|---|---|---|---|---|---|
| | Reagent Background Control Wells | | | | | | Plastic Background Control Wells | | | | | |
| A | (Media plus MTS, no cells) | | | | | | (Media only, no cells) | | | | | |
| B | Tox 1 | Cell | Drug 1 Low-Test | | | Tox 1 | Tox 2 | Drug 2 Low-Test | | | Cell | Tox 2 |
| | 0.32 μM | Control | 0.32 μM | | | 0.32 μM | 0.32 μM | 0.32 μM | | | Control | 0.32 μM |
| C | Tox 1 | | Drug 1 | | | Tox 1 | Tox 2 | Drug 2 | | | | Tox 2 |
| | 1.0 μM | | 1.0 μM | | | 1.0 μM | 1.0 μM | 1.0 μM | | | | 1.0 μM |
| D | Tox 1 | | Drug 1 | | | Tox 1 | Tox 2 | Drug 2 | | | | Tox 2 |
| | 3.2 μM | | 3.2 μM | | | 3.2 μM | 3.2 μM | 3.2 μM | | | | 3.2 μM |
| E | Tox 1 | Virus | Drug 1 | | | Tox 1 | Tox 2 | Drug 2 | | | Virus | Tox 2 |
| | 10 μM | Control | 10 μM | | | 10 μM | 10 μM | 10 μM | | | Control | 10 μM |
| F | Tox 1 | | Drug 1 | | | Tox 1 | Tox 2 | Drug 2 | | | | Tox 2 |
| | 32 μM | | 32 μM | | | 32 μM | 32 μM | 32 μM | | | | 32 μM |
| G | Tox 1 | | Drug 1 High-Test | | | Tox 1 | Tox 2 | Drug 2 High-Test | | | | Tox 2 |
| | 100 μM | | 100 μM | | | 100 μM | 100 μM | 100 μM | | | | 100 μM |
| H | Color 1 | Color 1 | Color 1 | Color 1 | Color 1 | Color 1 | Color 2 | Color 2 | Color 2 | Color 2 | Color 2 | Color 2 |
| | 100 μM | 32 μM | 10 μM | 3.2 μM | 1.0 μM | 0.32 μM | 100 μM | 32 μM | 10 μM | 3.2 μM | 1.0 μM | 0.32 μM |

Cells labeled as “Drug 1” or “Drug 2” = Cells + Virus + Drug (example dilution scheme indicated)
Cells labeled as “Tox 1” or “Tox 2” = Cells + Drug 1 or Drug 2, respectively (toxicity tested in duplicate)
Cells labeled as “Color 1” or “Color 2” = Media + Drug 1 or Drug 2, respectively (colorimetric background, no cells)

MTS Staining for Cell Viability

At assay termination (7 days post-infection), the assay plates are stained with the soluble tetrazolium-based dye MTS (3-(4,5-dimethylthiazol-2-yl)-5-(3-carboxymethoxyphenyl)-2-(4-sulfophenyl)-2H-tetrazolium; CellTiter®96 Reagent, Promega) to determine cell viability and quantify compound toxicity. MTS is metabolized by the mitochondrial enzymes of metabolically active cells to yield a soluble formazan product, allowing the rapid quantitative analysis of cell viability and compound cytotoxicity. This reagent is a stable, single solution that does not require preparation before use. At termination of the assay, 20-25 L of MTS reagent is added per well and the microtiter plates are then incubated for 4-6 hrs at 37° C., 5% CO₂ to assess cell viability. Adhesive plate sealers are used in place of the lids, the sealed plate is inverted several times to mix the soluble formazan product and the plate is read spectrophotometrically at 490/650 nm with a Molecular Devices Vmax or SpectraMax Plus plate reader.

Data Analysis

Using an in-house computer program % Cytopathic Effect (CPE) Reduction, % Cell Viability, IC₂₅, IC₅₀, IC₉₅, TC₂₅, TC₅₀, and TC₉₅ and other indices are calculated and the graphical results summary is displayed. Raw data for both antiviral activity and toxicity with a graphical representation of the data are provided in a printout summarizing the individual compound activity and reproduced in FIGS. 6A, 6B.

C. Description of the Rhinovirus Cytoprotection Assay in MRC-5 Cells

The Rhinovirus Cytoprotection assay uses MRC-5 cells and Rhinovirus strains 1B, 14, or 26. Briefly virus and cells are mixed in the presence of test compound and incubated for 7 days. The virus is pre-titered such that control wells exhibit 85 to 95% loss of cell viability due to virus replication. Therefore, antiviral effect or cytoprotection is observed when compounds prevent virus replication. Each assay plate contains cell control wells (cells only), virus control wells (cells plus virus), compound toxicity control wells (cells plus compound only), compound colorimetric control wells (compound only), as well as experimental wells (compound plus cells plus virus). Cytoprotection and compound cytotoxicity are assessed by MTS (CellTiter®96 Reagent, Promega, Madison Wis.) dye reduction. The % reduction in viral cytopathic effects (CPE) is determined and reported; IC₅₀ (concentration inhibiting virus replication by 50%), TC₅₀ (concentration resulting in 50% cell death) and a calculated TI (therapeutic index=TC₅₀/IC₅₀) are provided along with a graphical representation of the antiviral activity and compound cytotoxicity when compounds are tested in dose-response. Each assay includes enviroxime (ENV) as a positive control.

Cell Preparation

MRC-5 cells (Embryonal lung fibroblast, diploid, male, Human) were obtained from the American Type Culture Collection (ATCC, Rockville, Md.) and are grown in Eagle's Minimum Essential Medium with Earle's BSS (EMEM) supplemented with 10% fetal bovine serum (FBS), 0.1 mM non-essential amino acids, 1.0 mM sodium pyruvate, 2.0 mM L-Glutamine, 100 units/ml Penicillin and 100 μg/ml Streptomycin. Cells are sub-cultured twice a week at a split ratio of 1:2 using standard cell culture techniques. Total cell number and percent viability determinations are performed using a hemacytometer and trypan blue exclusion. Cell viability must be greater than 95% for the cells to be utilized in the assay. The cells are seeded in 96-well tissue culture plates the day before the assay at a concentration of 1×10⁴ cells/well.

Virus Preparation

The viruses used for this assay are Rhinovirus strains 1B, 14, or 26. These viruses were obtained from the American Type Culture Collection (ATCC) and was grown in MRC-5 cells for the production of stock virus pools. For each assay, a pre-titered aliquot of virus is removed from the freezer (−80° C.) and allowed to thaw slowly to room temperature in a biological safety cabinet. The virus is resuspended and diluted into tissue culture medium such that the amount of virus added to each well is the amount determined to give between 85 to 95% cell killing at 6-7 days post-infection.

Plate Format

A standardized plate format is used for cytoprotection assays. Each plate contains cell control wells (cells only), virus control wells (cells plus virus), drug cytotoxicity wells (cells plus drug only), drug colorimetric control wells (drug only), background control wells (media only), as well as experimental wells (drug plus cells plus virus). Samples are evaluated for antiviral efficacy with triplicate measurements using 6 concentrations at half-log dilutions (12 concentrations can also be performed) in order to determine IC₅₀ values and with duplicate measurements to determine cytotoxicity, if detectable. The Table below represents the standard plate format for testing compounds at 6 concentrations using a representative high-test concentration of 100 μM.

TABLE
Plate Format for Rhinovirus Cytoprotection Assays

| | 1 | 2 | 3 | 4 | 5 | 6 | 7 | 8 | 9 | 10 | 11 | 12 |
|---|---|---|---|---|---|---|---|---|---|---|---|---|
| | Reagent Background Control Wells | | | | | | Plastic Background Control Wells | | | | | |
| A | (Media plus MTS, no cells) | | | | | | (Media only, no cells) | | | | | |
| B | Tox 1 | Cell | Drug 1 Low-Test | | | Tox 1 | Tox 2 | Drug 2 Low-Test | | | Cell | Tox 2 |
| | 0.32 μM | Control | 0.32 μM | | | 0.32 μM | 0.32 μM | 0.32 μM | | | Control | 0.32 μM |
| C | Tox 1 | | Drug 1 | | | Tox 1 | Tox 2 | Drug 2 | | | | Tox 2 |
| | 1.0 μM | | 1.0 μM | | | 1.0 μM | 1.0 μM | 1.0 μM | | | | 1.0 μM |
| D | Tox 1 | | Drug 1 | | | Tox 1 | Tox 2 | Drug 2 | | | | Tox 2 |
| | 3.2 μM | | 3.2 μM | | | 3.2 μM | 3.2 μM | 3.2 μM | | | | 3.2 μM |
| E | Tox 1 | Virus | Drug 1 | | | Tox 1 | Tox 2 | Drug 2 | | | Virus | Tox 2 |
| | 10 μM | Control | 10 μM | | | 10 μM | 10 μM | 10 μM | | | Control | 10 μM |
| F | Tox 1 | | Drug 1 | | | Tox 1 | Tox 2 | Drug 2 | | | | Tox 2 |
| | 32 μM | | 32 μM | | | 32 μM | 32 μM | 32 μM | | | | 32 μM |
| G | Tox 1 | | Drug 1 High-Test | | | Tox 1 | Tox 2 | Drug 2 High-Test | | | | Tox 2 |
| | 100 μM | | 100 μM | | | 100 μM | 100 μM | 100 μM | | | | 100 μM |
| H | Color 1 | Color 1 | Color 1 | Color 1 | Color 1 | Color 1 | Color 2 | Color 2 | Color 2 | Color 2 | Color 2 | Color 2 |
| | 100 μM | 32 μM | 10 μM | 3.2 μM | 1.0 μM | 0.32 μM | 100 μM | 32 μM | 10 μM | 3.2 μM | 1.0 μM | 0.32 μM |

Cells labeled as “Drug 1” or “Drug 2” = Cells + Virus + Drug (example dilution scheme indicated)
Cells labeled as “Tox 1” or “Tox 2” = Cells + Drug 1 or Drug 2, respectively (toxicity tested in duplicate)
Cells labeled as “Color 1” or “Color 2” = Media + Drug 1 or Drug 2, respectively (colorimetric background, no cells)

MTS Staining for Cell Viability

At assay termination (7 days post-infection), the assay plates are stained with the soluble tetrazolium-based dye MTS (3-(4,5-dimethylthiazol-2-yl)-5-(3-carboxymethoxyphenyl)-2-(4-sulfophenyl)-2H-tetrazolium; CellTiter®96 Reagent, Promega) to determine cell viability and quantify compound toxicity. MTS is metabolized by the mitochondrial enzymes of metabolically active cells to yield a soluble formazan product, allowing the rapid quantitative analysis of cell viability and compound cytotoxicity. This reagent is a stable, single solution that does not require preparation before use. At termination of the assay, 20-25 L of MTS reagent is added per well and the microtiter plates are then incubated for 4-6 hrs at 37° C., 5% CO₂ to assess cell viability. Adhesive plate sealers are used in place of the lids, the sealed plate is inverted several times to mix the soluble formazan product and the plate is read spectrophotometrically at 490/650 nm with a Molecular Devices Vmax or SpectraMax Plus plate reader.

Data Analysis

Using an in-house computer program % Cytopathic Effect (CPE) Reduction, % Cell Viability, IC₂₅, IC₅₀, IC₉₅, TC₂₅, TC₅₀, and TC₉₅ and other indices are calculated and the graphical results summary is displayed. Raw data for both antiviral activity and toxicity with a graphical representation of the data are provided in a printout summarizing the individual compound activity and reproduced in FIGS. 7A, 7B.

D. Description of the Respiratory Syncytial Virus (RSV) Cytoprotection Assay Using A549 Cells

The Respiratory Syncytial Virus (RSV) Cytoprotection assay uses A549 cells and RSV strain Long. Briefly virus and cells are mixed in the presence of test compound and incubated for 7 days. The virus is pre-titered such that control wells exhibit 85 to 95% loss of cell viability due to virus replication. Therefore, antiviral effect or cytoprotection is observed when compounds prevent virus replication. Each assay plate contains cell control wells (cells only), virus control wells (cells plus virus), compound toxicity control wells (cells plus compound only), compound colorimetric control wells (compound only), as well as experimental wells (compound plus cells plus virus). Cytoprotection and compound cytotoxicity are assessed by MTS (CellTiter®96 Reagent, Promega, Madison Wis.) dye reduction. The % reduction in viral cytopathic effects (CPE) is determined and reported; IC₅₀ (concentration inhibiting virus replication by 50%), TC₅₀ (concentration resulting in 50% cell death) and a calculated TI (therapeutic index=TC₅₀/IC₅₀) are provided along with a graphical representation of the antiviral activity and compound cytotoxicity when compounds are tested in dose-response. Each assay includes ribavirin (RBV) as a positive control.

Cell Preparation

A549 cells (lung, epithelial, human) were obtained from the American Type Culture Collection (ATCC, Rockville, Md.) and are grown in F-12K Medium supplemented with 10% fetal bovine serum (FBS), 0.1 mM non-essential amino acids, 1.0 mM sodium pyruvate, 2.0 mM L-Glutamine, 100 units/ml Penicillin and 100 μg/ml Streptomycin. Cells are sub-cultured twice a week at a split ratio of 1:5 to 1:10 using standard cell culture techniques. Total cell number and percent viability determinations are performed using a hemacytometer and trypan blue exclusion. Cell viability must be greater than 95% for the cells to be utilized in the assay. The cells are seeded in 96-well tissue culture plates the day before the assay at a concentration of 5×10³ cells/well.

Virus Preparation

The virus used for this assay is RSV strain Long. This virus was obtained from the American Type Culture Collection (ATCC) and was grown in Vero cells for the production of stock virus pools. For each assay, a pre-titered aliquot of virus is removed from the freezer (−80° C.) and allowed to thaw slowly to room temperature in a biological safety cabinet. The virus is resuspended and diluted into tissue culture medium such that the amount of virus added to each well is the amount determined to give between 85 to 95% cell killing at 6-7 days post-infection.

Plate Format

A standardized plate format is used for cytoprotection assays. Each plate contains cell control wells (cells only), virus control wells (cells plus virus), drug cytotoxicity wells (cells plus drug only), drug colorimetric control wells (drug only), background control wells (media only), as well as experimental wells (drug plus cells plus virus). Samples are evaluated for antiviral efficacy with triplicate measurements using 6 concentrations at half-log dilutions (12 concentrations can also be performed) in order to determine IC₅₀ values and with duplicate measurements to determine cytotoxicity, if detectable. The Table below represents the standard plate format for testing compounds at 6 concentrations using a representative high-test concentration of 100 μM.

TABLE
Plate Format for RSV Cytoprotection Assays

| | 1 | 2 | 3 | 4 | 5 | 6 | 7 | 8 | 9 | 10 | 11 | 12 |
|---|---|---|---|---|---|---|---|---|---|---|---|---|
| | Reagent Background Control Wells | | | | | | Plastic Background Control Wells | | | | | |
| A | (Media plus MTS, no cells) | | | | | | (Media only, no cells) | | | | | |
| B | Tox 1 | Cell | Drug 1 Low-Test | | | Tox 1 | Tox 2 | Drug 2 Low-Test | | | Cell | Tox 2 |
| | 0.32 μM | Control | 0.32 μM | | | 0.32 μM | 0.32 μM | 0.32 μM | | | Control | 0.32 μM |
| C | Tox 1 | | Drug 1 | | | Tox 1 | Tox 2 | Drug 2 | | | | Tox 2 |
| | 1.0 μM | | 1.0 μM | | | 1.0 μM | 1.0 μM | 1.0 μM | | | | 1.0 μM |
| D | Tox 1 | | Drug 1 | | | Tox 1 | Tox 2 | Drug 2 | | | | Tox 2 |
| | 3.2 μM | | 3.2 μM | | | 3.2 μM | 3.2 μM | 3.2 μM | | | | 3.2 μM |
| E | Tox 1 | Virus | Drug 1 | | | Tox 1 | Tox 2 | Drug 2 | | | Virus | Tox 2 |
| | 10 μM | Control | 10 μM | | | 10 μM | 10 μM | 10 μM | | | Control | 10 μM |
| F | Tox 1 | | Drug 1 | | | Tox 1 | Tox 2 | Drug 2 | | | | Tox 2 |
| | 32 μM | | 32 μM | | | 32 μM | 32 μM | 32 μM | | | | 32 μM |
| G | Tox 1 | | Drug 1 High-Test | | | Tox 1 | Tox 2 | Drug 2 High-Test | | | | Tox 2 |
| | 100 μM | | 100 μM | | | 100 μM | 100 μM | 100 μM | | | | 100 μM |
| H | Color 1 | Color 1 | Color 1 | Color 1 | Color 1 | Color 1 | Color 2 | Color 2 | Color 2 | Color 2 | Color 2 | Color 2 |
| | 100 μM | 32 μM | 10 μM | 3.2 μM | 1.0 μM | 0.32 μM | 100 μM | 32 μM | 10 μM | 3.2 μM | 1.0 μM | 0.32 μM |

Cells labeled as “Drug 1” or “Drug 2” = Cells + Virus + Drug (example dilution scheme indicated)
Cells labeled as “Tox 1” or “Tox 2” = Cells + Drug 1 or Drug 2, respectively (toxicity tested in duplicate)
Cells labeled as “Color 1” or “Color 2” = Media + Drug 1 or Drug 2, respectively (colorimetric background, no cells)

MTS Staining for Cell Viability

At assay termination (7 days post-infection), the assay plates are stained with the soluble tetrazolium-based dye MTS (3-(4,5-dimethylthiazol-2-yl)-5-(3-carboxymethoxyphenyl)-2-(4-sulfophenyl)-2H-tetrazolium; CellTiter®96 Reagent, Promega) to determine cell viability and quantify compound toxicity. MTS is metabolized by the mitochondrial enzymes of metabolically active cells to yield a soluble formazan product, allowing the rapid quantitative analysis of cell viability and compound cytotoxicity. This reagent is a stable, single solution that does not require preparation before use. At termination of the assay, 20-25 L of MTS reagent is added per well and the microtiter plates are then incubated for 4-6 hrs at 37° C., 5% CO₂ to assess cell viability. Adhesive plate sealers are used in place of the lids, the sealed plate is inverted several times to mix the soluble formazan product and the plate is read spectrophotometrically at 490/650 nm with a Molecular Devices Vmax or SpectraMax Plus plate reader.

Data Analysis

Using an in-house computer program % Cytopathic Effect (CPE) Reduction, % Cell Viability, IC₂₅, IC₅₀, IC₉₅, TC₂₅, TC₅₀, and TC₉₅ and other indices are calculated and the graphical results summary is displayed. Raw data for both antiviral activity and toxicity with a graphical representation of the data are provided in a printout summarizing the individual compound activity and reproduced in FIGS. 8A, 8B.

E. Description of the Influenza a Cytoprotection Assay

The Influenza A Cytoprotection assay uses MDCK cells and Influenza A strains A/Victoria/3/75 (H3N2), A/Hong Kong/8/68 (H3N2), A/PR/8/34 (H1N1), or A/WS/33 (H1N1). Briefly virus and cells are mixed in the presence of test compound and incubated for 7 days. The virus is pre-titered such that control wells exhibit 85 to 95% loss of cell viability due to virus replication. Therefore, antiviral effect or cytoprotection is observed when compounds prevent virus replication. Each assay plate contains cell control wells (cells only), virus control wells (cells plus virus), compound toxicity control wells (cells plus compound only), compound colorimetric control wells (compound only), as well as experimental wells (compound plus cells plus virus). Cytoprotection and compound cytotoxicity are assessed by MTS (CellTiter®96 Reagent, Promega, Madison Wis.) dye reduction. The % reduction in viral cytopathic effects (CPE) is determined and reported; IC₅₀ (concentration inhibiting virus replication by 50%), TC₅₀ (concentration resulting in 50% cell death) and a calculated TI (therapeutic index=TC₅₀/IC₅₀) are provided along with a graphical representation of the antiviral activity and compound cytotoxicity when compounds are tested in dose-response. Each assay includes ribavirin (RBV) as a positive control.

Cell Preparation

MDCK cells (Kidney, dog, Canis familiaris) were obtained from the American Type Culture Collection (ATCC, Rockville, Md.) and are grown in Eagle's Minimum Essential Medium with Earle's BSS (EMEM) supplemented with 10% fetal bovine serum (FBS), 0.1 mM non-essential amino acids, 1.0 mM sodium pyruvate, 2.0 mM L-Glutamine, 100 units/ml Penicillin and 100 μg/ml Streptomycin. Cells are sub-cultured twice a week at a split ratio of 1:4 using standard cell culture techniques. Total cell number and percent viability determinations are performed using a hemacytometer and trypan blue exclusion. Cell viability must be greater than 95% for the cells to be utilized in the assay. The cells are seeded in 96-well tissue culture plates the day before the assay at a concentration of 1×10⁴ cells/well.

Virus Preparation

The viruses used for this assay are Influenza A strains A/Victoria/3/75 (H3N2), A/Hong Kong/8/68 (H3N2), A/PR/8/34 (H1N1), or A/WS/33 (H1N1). These viruses were obtained from the American Type Culture Collection (ATCC) and were grown in MDCK cells for the production of stock virus pools. For each assay, a pre-titered aliquot of virus is removed from the freezer (−80° C.) and allowed to thaw slowly to room temperature in a biological safety cabinet. The virus is resuspended and diluted into tissue culture medium such that the amount of virus added to each well is the amount determined to give between 85 to 95% cell killing at 6-7 days post-infection.

Plate Format

A standardized plate format is used for cytoprotection assays. Each plate contains cell control wells (cells only), virus control wells (cells plus virus), drug cytotoxicity wells (cells plus drug only), drug colorimetric control wells (drug only), background control wells (media only), as well as experimental wells (drug plus cells plus virus). Samples are evaluated for antiviral efficacy with triplicate measurements using 6 concentrations at half-log dilutions (12 concentrations can also be performed) in order to determine IC₅₀ values and with duplicate measurements to determine cytotoxicity, if detectable. The Table below represents the standard plate format for testing compounds at 6 concentrations using a representative high-test concentration of 100 μM.

TABLE
Plate Format for Influenza A Cytoprotection Assays

| | 1 | 2 | 3 | 4 | 5 | 6 | 7 | 8 | 9 | 10 | 11 | 12 |
|---|---|---|---|---|---|---|---|---|---|---|---|---|
| | Reagent Background Control Wells | | | | | | Plastic Background Control Wells | | | | | |
| A | (Media plus MTS, no cells) | | | | | | (Media only, no cells) | | | | | |
| B | Tox 1 | Cell | Drug 1 Low-Test | | | Tox 1 | Tox 2 | Drug 2 Low-Test | | | Cell | Tox 2 |
| | 0.32 μM | Control | 0.32 μM | | | 0.32 μM | 0.32 μM | 0.32 μM | | | Control | 0.32 μM |
| C | Tox 1 | | Drug 1 | | | Tox 1 | Tox 2 | Drug 2 | | | | Tox 2 |
| | 1.0 μM | | 1.0 μM | | | 1.0 μM | 1.0 μM | 1.0 μM | | | | 1.0 μM |
| D | Tox 1 | | Drug 1 | | | Tox 1 | Tox 2 | Drug 2 | | | | Tox 2 |
| | 3.2 μM | | 3.2 μM | | | 3.2 μM | 3.2 μM | 3.2 μM | | | | 3.2 μM |
| E | Tox 1 | Virus | Drug 1 | | | Tox 1 | Tox 2 | Drug 2 | | | Virus | Tox 2 |
| | 10 μM | Control | 10 μM | | | 10 μM | 10 μM | 10 μM | | | Control | 10 μM |
| F | Tox 1 | | Drug 1 | | | Tox 1 | Tox 2 | Drug 2 | | | | Tox 2 |
| | 32 μM | | 32 μM | | | 32 μM | 32 μM | 32 μM | | | | 32 μM |
| G | Tox 1 | | Drug 1 High-Test | | | Tox 1 | Tox 2 | Drug 2 High-Test | | | | Tox 2 |
| | 100 μM | | 100 μM | | | 100 μM | 100 μM | 100 μM | | | | 100 μM |
| H | Color 1 | Color 1 | Color 1 | Color 1 | Color 1 | Color 1 | Color 2 | Color 2 | Color 2 | Color 2 | Color 2 | Color 2 |
| | 100 μM | 32 μM | 10 μM | 3.2 μM | 1.0 μM | 0.32 μM | 100 μM | 32 μM | 10 μM | 3.2 μM | 1.0 μM | 0.32 μM |

Cells labeled as “Drug 1” or “Drug 2” = Cells + Virus + Drug (example dilution scheme indicated)
Cells labeled as “Tox 1” or “Tox 2” = Cells + Drug 1 or Drug 2, respectively (toxicity tested in duplicate)
Cells labeled as “Color 1” or “Color 2” = Media + Drug 1 or Drug 2, respectively (colorimetric background, no cells)

MTS Staining for Cell Viability

At assay termination (7 days post-infection), the assay plates are stained with the soluble tetrazolium-based dye MTS (3-(4,5-dimethylthiazol-2-yl)-5-(3-carboxymethoxyphenyl)-2-(4-sulfophenyl)-2H-tetrazolium; CellTiter®96 Reagent, Promega) to determine cell viability and quantify compound toxicity. MTS is metabolized by the mitochondrial enzymes of metabolically active cells to yield a soluble formazan product, allowing the rapid quantitative analysis of cell viability and compound cytotoxicity. This reagent is a stable, single solution that does not require preparation before use. At termination of the assay, 20-25 L of MTS reagent is added per well and the microtiter plates are then incubated for 4-6 hrs at 37° C., 5% CO₂ to assess cell viability. Adhesive plate sealers are used in place of the lids, the sealed plate is inverted several times to mix the soluble formazan product and the plate is read spectrophotometrically at 490/650 nm with a Molecular Devices Vmax or SpectraMax Plus plate reader.

Data Analysis

Using an in-house computer program % Cytopathic Effect (CPE) Reduction, % Cell Viability, IC₂₅, IC₅₀, IC₉₅, TC₂₅, TC₅₀, and TC₉₅ and other indices are calculated and the graphical results summary is displayed. Raw data for both antiviral activity and toxicity with a graphical representation of the data are provided in a printout summarizing the individual compound activity and reproduced in FIGS. 9A, 9B.

F. Description of the Coxsackie A Virus Cytoprotection Assay

The Coxsackie A virus Cytoprotection assay uses MRC-5 cells and Coxsackie A strains A7 or A21. Briefly virus and cells are mixed in the presence of test compound and incubated for 7 days. The virus is pre-titered such that control wells exhibit 85 to 95% loss of cell viability due to virus replication. Therefore, antiviral effect or cytoprotection is observed when compounds prevent virus replication. Each assay plate contains cell control wells (cells only), virus control wells (cells plus virus), compound toxicity control wells (cells plus compound only), compound colorimetric control wells (compound only), as well as experimental wells (compound plus cells plus virus). Cytoprotection and compound cytotoxicity are assessed by MTS (CellTiter®96 Reagent, Promega, Madison Wis.) dye reduction. The % reduction in viral cytopathic effects (CPE) is determined and reported; IC₅₀ (concentration inhibiting virus replication by 50%), TC₅₀ (concentration resulting in 50% cell death) and a calculated TI (therapeutic index=TC₅₀/IC₅₀) are provided along with a graphical representation of the antiviral activity and compound cytotoxicity when compounds are tested in dose-response. Each assay includes ribavirin (RBV) as a positive control.

Cell Preparation

MRC-5 cells (Embryonal lung fibroblast, diploid, male, Human) were obtained from the American Type Culture Collection (ATCC, Rockville, Md.) and are grown in Eagle's Minimum Essential Medium with Earle's BSS (EMEM) supplemented with 10% fetal bovine serum (FBS), 0.1 mM non-essential amino acids, 1.0 mM sodium pyruvate, 2.0 mM L-Glutamine, 100 units/ml Penicillin and 100 μg/ml Streptomycin. Cells are sub-cultured twice a week at a split ratio of 1:2 using standard cell culture techniques. Total cell number and percent viability determinations are performed using a hemacytometer and trypan blue exclusion. Cell viability must be greater than 95% for the cells to be utilized in the assay. The cells are seeded in 96-well tissue culture plates the day before the assay at a concentration of 1×10⁴ cells/well.

Virus Preparation

The viruses used for this assay are Coxsackie A strains A7 or A21. These viruses were obtained from the American Type Culture Collection (ATCC) and were grown in MRC-5 cells for the production of stock virus pools. For each assay, a pre-titered aliquot of virus is removed from the freezer (−80° C.) and allowed to thaw slowly to room temperature in a biological safety cabinet. The virus is resuspended and diluted into tissue culture medium such that the amount of virus added to each well is the amount determined to give between 85 to 95% cell killing at 7 days post-infection.

Plate Format

A standardized plate format is used for cytoprotection assays. Each plate contains cell control wells (cells only), virus control wells (cells plus virus), drug cytotoxicity wells (cells plus drug only), drug colorimetric control wells (drug only), background control wells (media only), as well as experimental wells (drug plus cells plus virus). Samples are evaluated for antiviral efficacy with triplicate measurements using 6 concentrations at half-log dilutions (12 concentrations can also be performed) in order to determine IC₅₀ values and with duplicate measurements to determine cytotoxicity, if detectable. The Table below represents the standard plate format for testing compounds at 6 concentrations using a representative high-test concentration of 100 μM.

TABLE
Plate Format for Coxsackie Virus Cytoprotection Assays

| | 1 | 2 | 3 | 4 | 5 | 6 | 7 | 8 | 9 | 10 | 11 | 12 |
|---|---|---|---|---|---|---|---|---|---|---|---|---|
| | Reagent Background Control Wells | | | | | | Plastic Background Control Wells | | | | | |
| A | (Media plus MTS, no cells) | | | | | | (Media only, no cells) | | | | | |
| B | Tox 1 | Cell | Drug 1 Low-Test | | | Tox 1 | Tox 2 | Drug 2 Low-Test | | | Cell | Tox 2 |
| | 0.32 μM | Control | 0.32 μM | | | 0.32 μM | 0.32 μM | 0.32 μM | | | Control | 0.32 μM |
| C | Tox 1 | | Drug 1 | | | Tox 1 | Tox 2 | Drug 2 | | | | Tox 2 |
| | 1.0 μM | | 1.0 μM | | | 1.0 μM | 1.0 μM | 1.0 μM | | | | 1.0 μM |
| D | Tox 1 | | Drug 1 | | | Tox 1 | Tox 2 | Drug 2 | | | | Tox 2 |
| | 3.2 μM | | 3.2 μM | | | 3.2 μM | 3.2 μM | 3.2 μM | | | | 3.2 μM |
| E | Tox 1 | Virus | Drug 1 | | | Tox 1 | Tox 2 | Drug 2 | | | Virus | Tox 2 |
| | 10 μM | Control | 10 μM | | | 10 μM | 10 μM | 10 μM | | | Control | 10 μM |
| F | Tox 1 | | Drug 1 | | | Tox 1 | Tox 2 | Drug 2 | | | | Tox 2 |
| | 32 μM | | 32 μM | | | 32 μM | 32 μM | 32 μM | | | | 32 μM |
| G | Tox 1 | | Drug 1 High-Test | | | Tox 1 | Tox 2 | Drug 2 High-Test | | | | Tox 2 |
| | 100 μM | | 100 μM | | | 100 μM | 100 μM | 100 μM | | | | 100 μM |
| H | Color 1 | Color 1 | Color 1 | Color 1 | Color 1 | Color 1 | Color 2 | Color 2 | Color 2 | Color 2 | Color 2 | Color 2 |
| | 100 μM | 32 μM | 10 μM | 3.2 μM | 1.0 μM | 0.32 μM | 100 μM | 32 μM | 10 μM | 3.2 μM | 1.0 μM | 0.32 μM |

Cells labeled as “Drug 1” or “Drug 2” = Cells + Virus + Drug (example dilution scheme indicated)
Cells labeled as “Tox 1” or “Tox 2” = Cells + Drug 1 or Drug 2, respectively (toxicity tested in duplicate)
Cells labeled as “Color 1” or “Color 2” = Media + Drug 1 or Drug 2, respectively (colorimetric background, no cells).

MTS Staining for Cell Viability

At assay termination (7 days post-infection), the assay plates are stained with the soluble tetrazolium-based dye MTS (3-(4,5-dimethylthiazol-2-yl)-5-(3-carboxymethoxyphenyl)-2-(4-sulfophenyl)-2H-tetrazolium; CellTiter®96 Reagent, Promega) to determine cell viability and quantify compound toxicity. MTS is metabolized by the mitochondrial enzymes of metabolically active cells to yield a soluble formazan product, allowing the rapid quantitative analysis of cell viability and compound cytotoxicity. This reagent is a stable, single solution that does not require preparation before use. At termination of the assay, 20-25 L of MTS reagent is added per well and the microtiter plates are then incubated for 4-6 hrs at 37° C., 5% CO₂ to assess cell viability. Adhesive plate sealers are used in place of the lids, the sealed plate is inverted several times to mix the soluble formazan product and the plate is read spectrophotometrically at 490/650 nm with a Molecular Devices Vmax or SpectraMax Plus plate reader.

Data Analysis

Using an in-house computer program % Cytopathic Effect (CPE) Reduction, % Cell Viability, IC₂₅, IC₅₀, IC₉₅, TC₂₅, TC₅₀, and TC₉₅ and other indices are calculated and the graphical results summary is displayed. Raw data for both antiviral activity and toxicity with a graphical representation of the data are provided in a printout summarizing the individual compound activity and reproduced in FIGS. 10A, 10B.

G. Description of HSV Cytoprotection Assay

The HSV Cytoprotection assay uses Vero cells and HSV-1 strain HF or HSV-2 strain MS. Briefly, virus and cells are mixed in the presence of test compound and incubated for 5 days. The virus is pre-titered such that control wells exhibit 85 to 95% loss of cell viability due to virus replication. Therefore, antiviral effect, or cytoprotection, is observed when compounds prevent virus replication. Each assay plate contains cell control wells (cells only), virus control wells (cells plus virus), compound toxicity control wells (cells plus compound only), compound colorimetric control wells (compound only), as well as experimental wells (compound plus cells plus virus). Cytoprotection and compound cytotoxicity are assessed by MTS (CellTiter®96 Reagent, Promega, Madison Wis.) dye reduction. The % reduction in viral cytopathic effects (CPE) is determined and reported; IC₅₀ (concentration inhibiting virus replication by 50%), TC₅₀ (concentration resulting in 50% cell death) and a calculated TI (therapeutic index=TC₅₀/IC₅₀) are provided along with a graphical representation of the antiviral activity and compound cytotoxicity when compounds are tested in dose-response. Each assay includes acyclovir (ACV) as a positive control.

Cell Preparation

Vero cells (Kidney, African green monkey, Cercopithecus aethiops) were obtained from the American Type Culture Collection (ATCC, Rockville, Md.) and are grown in Dulbecco's Modified Eagle's Medium (DMEM) supplemented with 10% fetal bovine serum (FBS), 2.0 mM L-Glutamine, 100 units/ml Penicillin and 100 ug/ml Streptomycin. Cells are sub-cultured twice a week at a split ratio of 1:10 using standard cell culture techniques. Total cell number and percent viability determinations are performed using a hemacytometer and trypan blue exclusion. Cell viability must be greater than 95% for the cells to be utilized in the assay. The cells are seeded in 96-well tissue culture plates the day before the assay at a concentration of 1×10⁴ cells/well.

Virus Preparation

The standard viruses used for this assay are HSV-1 strain HF and HSV-2 strain MS (other viruses available upon request). These viruses were obtained from the American Type Culture Collection (ATCC) and were grown in Vero cells for the production of stock virus pools. For each assay, a pre-titered aliquot of virus is removed from the freezer (−80° C.) and allowed to thaw slowly to room temperature in a biological safety cabinet. The virus is resuspended and diluted into tissue culture medium such that the amount of virus added to each well is the amount determined to give between 85 to 95% cell killing at 5 days post-infection.

Plate Format

A standardized plate format is used for cytoprotection assays. Each plate contains cell control wells (cells only), virus control wells (cells plus virus), drug cytotoxicity wells (cells plus drug only), drug colorimetric control wells (drug only), background control wells (media only), as well as experimental wells (drug plus cells plus virus). Samples are evaluated for antiviral efficacy with triplicate measurements using 6 concentrations at half-log dilutions (12 concentrations can also be performed) in order to determine IC₅₀ values and with duplicate measurements to determine cytotoxicity, if detectable. The Table below represents the standard plate format for testing compounds at 6 concentrations using a representative high-test concentration of 100 μM.

TABLE
Plate Format for HSV Cytoprotection Assays

| | 1 | 2 | 3 | 4 | 5 | 6 | 7 | 8 | 9 | 10 | 11 | 12 |
|---|---|---|---|---|---|---|---|---|---|---|---|---|
| | Reagent Background Control Wells | | | | | | Plastic Background Control Wells | | | | | |
| A | (Media plus MTS, no cells) | | | | | | (Media only, no cells) | | | | | |
| B | Tox 1 | Cell | Drug 1 Low-Test | | | Tox 1 | Tox 2 | Drug 2 Low-Test | | | Cell | Tox 2 |
| | 0.32 μM | Control | 0.32 μM | | | 0.32 μM | 0.32 μM | 0.32 μM | | | Control | 0.32 μM |
| C | Tox 1 | | Drug 1 | | | Tox 1 | Tox 2 | Drug 2 | | | | Tox 2 |
| | 1.0 μM | | 1.0 μM | | | 1.0 μM | 1.0 μM | 1.0 μM | | | | 1.0 μM |
| D | Tox 1 | | Drug 1 | | | Tox 1 | Tox 2 | Drug 2 | | | | Tox 2 |
| | 3.2 μM | | 3.2 μM | | | 3.2 μM | 3.2 μM | 3.2 μM | | | | 3.2 μM |
| E | Tox 1 | Virus | Drug 1 | | | Tox 1 | Tox 2 | Drug 2 | | | Virus | Tox 2 |
| | 10 μM | Control | 10 μM | | | 10 μM | 10 μM | 10 μM | | | Control | 10 μM |
| F | Tox 1 | | Drug 1 | | | Tox 1 | Tox 2 | Drug 2 | | | | Tox 2 |
| | 32 μM | | 32 μM | | | 32 μM | 32 μM | 32 μM | | | | 32 μM |
| G | Tox 1 | | Drug 1 High-Test | | | Tox 1 | Tox 2 | Drug 2 High-Test | | | | Tox 2 |
| | 100 μM | | 100 μM | | | 100 μM | 100 μM | 100 μM | | | | 100 μM |
| H | Color 1 | Color 1 | Color 1 | Color 1 | Color 1 | Color 1 | Color 2 | Color 2 | Color 2 | Color 2 | Color 2 | Color 2 |
| | 100 μM | 32 μM | 10 μM | 3.2 μM | 1.0 μM | 0.32 μM | 100 μM | 32 μM | 10 μM | 3.2 μM | 1.0 μM | 0.32 μM |

Cells labeled as “Drug 1” or “Drug 2” = Cells + Virus + Drug (example dilution scheme indicated)
Cells labeled as “Tox 1” or “Tox 2” = Cells + Drug 1 or Drug 2, respectively (toxicity tested in duplicate)
Cells labeled as “Color 1” or “Color 2” = Media + Drug 1 or Drug 2, respectively (colorimetric background, no cells)

MTS Staining for Cell Viability

At assay termination (5 days post-infection), the assay plates are stained with the soluble tetrazolium-based dye MTS (3-(4,5-dimethylthiazol-2-yl)-5-(3-carboxymethoxyphenyl)-2-(4-sulfophenyl)-2H-tetrazolium; CellTiter®96 Reagent, Promega) to determine cell viability and quantify compound toxicity. MTS is metabolized by the mitochondrial enzymes of metabolically active cells to yield a soluble formazan product, allowing the rapid quantitative analysis of cell viability and compound cytotoxicity. This reagent is a stable, single solution that does not require preparation before use. At termination of the assay, 20-25 L of MTS reagent is added per well and the microtiter plates are then incubated for 4-6 hrs at 37° C., 5% CO₂ to assess cell viability. Adhesive plate sealers are used in place of the lids, the sealed plate is inverted several times to mix the soluble formazan product and the plate is read spectrophotometrically at 490/650 nm with a Molecular Devices Vmax or SpectraMax Plus plate reader.

Data Analysis

Using an in-house computer program % Cytopathic Effect (CPE) Reduction, % Cell Viability, IC₂₅, IC₅₀, IC₉₅, TC₂₅, TC₅₀, and TC₉₅ and other indices are calculated and the graphical results summary is displayed. Raw data for both antiviral activity and toxicity with a graphical representation of the data are provided in a printout summarizing the individual compound activity and reproduced in FIGS. 11A, B.

H. Description of the HIV-1 Cytoprotection Assay

The HIV Cytoprotection assay uses CEM-SS cells and the IIIB or RF strain of HIV-1. Briefly, virus and cells are mixed in the presence of test compound and incubated for 6 days. The virus is pre-titered such that control wells exhibit 70 to 95% loss of cell viability due to virus replication. Therefore, antiviral effect or cytoprotection is observed when compounds prevent virus replication. Each assay plate contains cell control wells (cells only), virus control wells (cells plus virus), compound toxicity control wells (cells plus compound only), compound colorimetric control wells (compound only), as well as experimental wells (compound plus cells plus virus). Cytoprotection and compound cytotoxicity are assessed by MTS (CellTiter®96 Reagent, Promega, Madison Wis.) dye reduction. The % reduction in viral cytopathic effects (CPE) is determined and reported; IC₅₀ (concentration inhibiting virus replication by 50%), TC₅₀ (concentration resulting in 50% cell death) and a calculated TI (therapeutic index TC₅₀/IC₅₀) are provided along with a graphical representation of the antiviral activity and compound cytotoxicity when compounds are tested in dose-response. Each assay includes the HIV reverse transcriptase inhibitor AZT as a positive control. Other controls can be added upon request.

Cell Preparation

CEM-SS cells were obtained from the NIH AIDS Research and Reference Reagent Program and are routinely passaged in T-75 flasks using standard tissue culture techniques based on the specifications provided by the supplier. On the day preceding the assay, the cells are split 1:2 to assure they are in an exponential growth phase at the time of infection. Total cell number and percent viability determinations are performed using a hemacytometer and trypan blue exclusion. Cell viability must be greater than 95% for the cells to be utilized in the assay. The cells are re-suspended at 5×10⁴ cells/mL in tissue culture medium and added to the drug-containing 96-well microtiter plates in a volume of 50 μl.

Virus Preparation

The viruses used for this assay are CXCR4-tropic laboratory virus strains. The most commonly used strains are HIV-1_{RF} and HIV-1_{IIIB} (each obtained from the NIH AIDS Research and Reference Reagent Program). For each assay, a pre-titered aliquot of virus is removed from the freezer (−80° C.) and allowed to thaw slowly to room temperature in a biological safety cabinet. The virus is re-suspended and diluted into tissue culture medium such that the amount of virus added to each well in a volume of 50 μl is the amount determined to give between 85 to 95% cell killing at 6 days post-infection. TCID₅₀ calculations by endpoint titration in the assay indicates that the multiplicity of infection of these assays is approximately 0.01.

Plate Format

A standardized plate format is used for cytoprotection assays. Each plate contains cell control wells (cells only), virus control wells (cells plus virus), drug cytotoxicity wells (cells plus drug only), drug colorimetric control wells (drug only), background control wells (media only), as well as experimental wells (drug plus cells plus virus). Samples are evaluated for antiviral efficacy with triplicate measurements using 6 concentrations at half-log dilutions (12 concentrations can also be performed) in order to determine IC₅₀ values and with duplicate measurements to determine cytotoxicity, if detectable. The Table below represents the standard plate format for testing compounds at 6 concentrations using a representative high-test concentration of 100 μM.

TABLE
Plate Format for HIV-1 Cytoprotection Assays

| | 1 | 2 | 3 | 4 | 5 | 6 | 7 | 8 | 9 | 10 | 11 | 12 |
|---|---|---|---|---|---|---|---|---|---|---|---|---|
| | Reagent Background Control Wells | | | | | | Plastic Background Control Wells | | | | | |
| A | (Media plus MTS, no cells) | | | | | | (Media only, no cells) | | | | | |
| B | Tox 1 | Cell | Drug 1 Low-Test | | | Tox 1 | Tox 2 | Drug 2 Low-Test | | | Cell | Tox 2 |
| | 0.32 μM | Control | 0.32 μM | | | 0.32 μM | 0.32 μM | 0.32 μM | | | Control | 0.32 μM |
| C | Tox 1 | | Drug 1 | | | Tox 1 | Tox 2 | Drug 2 | | | | Tox 2 |
| | 1.0 μM | | 1.0 μM | | | 1.0 μM | 1.0 μM | 1.0 μM | | | | 1.0 μM |
| D | Tox 1 | | Drug 1 | | | Tox 1 | Tox 2 | Drug 2 | | | | Tox 2 |
| | 3.2 μM | | 3.2 μM | | | 3.2 μM | 3.2 μM | 3.2 μM | | | | 3.2 μM |
| E | Tox 1 | Virus | Drug 1 | | | Tox 1 | Tox 2 | Drug 2 | | | Virus | Tox 2 |
| | 10 μM | Control | 10 μM | | | 10 μM | 10 μM | 10 μM | | | Control | 10 μM |
| F | Tox 1 | | Drug 1 | | | Tox 1 | Tox 2 | Drug 2 | | | | Tox 2 |
| | 32 μM | | 32 μM | | | 32 μM | 32 μM | 32 μM | | | | 32 μM |
| G | Tox 1 | | Drug 1 High-Test | | | Tox 1 | Tox 2 | Drug 2 High-Test | | | | Tox 2 |
| | 100 μM | | 100 μM | | | 100 μM | 100 μM | 100 μM | | | | 100 μM |
| H | Color 1 | Color 1 | Color 1 | Color 1 | Color 1 | Color 1 | Color 2 | Color 2 | Color 2 | Color 2 | Color 2 | Color 2 |
| | 100 μM | 32 μM | 10 μM | 3.2 μM | 1.0 μM | 0.32 μM | 100 μM | 32 μM | 10 μM | 3.2 μM | 1.0 μM | 0.32 μM |

Cells labeled as “Drug 1” or “Drug 2” = Cells + Virus + Drug (example dilution scheme indicated)
Cells labeled as “Tox 1” or “Tox 2” = Cells + Drug 1 or Drug 2, respectively (toxicity tested in duplicate)
Cells labeled as “Color 1” or “Color 2” = Media + Drug 1 or Drug 2, respectively (colorimetric background, no cells)

MTS Staining for Cell Viability

At assay termination (6 days post-infection), the assay plates are stained with the soluble tetrazolium-based dye MTS (3-(4,5-dimethylthiazol-2-yl)-5-(3-carboxymethoxyphenyl)-2-(4-sulfophenyl)-2H-tetrazolium; CellTiter 96 Reagent, Promega) to determine cell viability and quantify compound toxicity. MTS is metabolized by the mitochondrial enzymes of metabolically active cells to yield a soluble formazan product, allowing the rapid quantitative analysis of cell viability and compound cytotoxicity. This reagent is a stable, single solution that does not require preparation before use. At termination of the assay, 20-25 L of MTS reagent is added per well and the microtiter plates are then incubated for 4-6 hrs at 37° C., 5% CO₂ to assess cell viability. Adhesive plate sealers are used in place of the lids, the sealed plate is inverted several times to mix the soluble formazan product and the plate is read spectrophotometrically at 490/650 nm with a Molecular Devices Vmax or SpectraMax Plus plate reader.

Data Analysis

Using an in-house computer program % Cytopathic Effect (CPE) Reduction, % Cell Viability, IC₂₅, IC₅₀, IC₉₅, TC₂₅, TC₅₀, and TC₉₅ and other indices are calculated and the graphical results summary is displayed. Raw data for both antiviral activity and toxicity with a graphical representation of the data are provided in a printout summarizing the individual compound activity and reproduced in FIGS. 12A, 12B.

### SUMMARY OF RESULTS

The table below shows the incredible and unexpected results of assay testing of Macrocystis pyrifera derived High-Purity Fucoidan in comparison to the best known agents for treating the viruses identified below. The chart below is a summation of the test results shown in FIGS. 6A, 6B-12A, 12B.

TABLE
Antiviral Evaluation of Macrocystis pyrifera 78% Fucoidan

| | | | | | | | | Antiviral |
|---|---|---|---|---|---|---|---|---|
| Virus | Compound | High-Test | | IC₅₀ | | TC₅₀ | | Index |
| DENV2 | Fucoidan | 100 | μg/ml | 6.91 | μg/ml | >100 | μg/ml | >14.5 |
| Strain New | Ribavirin | 200 | μg/ml | 30.7 | μg/ml | >200 | μg/ml | >6.52 |
| Guinea C | | | | | | | | |
| HSV-1 | Fucoidan | 100 | μg/ml | >100 | μg/ml | >100 | μg/ml | N/A |
| Strain HF | Acyclovir | 100 | μM | 6.92 | μM | >100 | μM | >14.5 |
| Influenza A | Fucoidan | 100 | μg/ml | >100 | μg/ml | >100 | μg/ml | N/A |
| Strain Victoria | Ribavirin | 100 | μg/ml | 4.45 | μg/ml | >100 | μg/ml | >22.5 |
| RSV | Fucoidan | 100 | μg/ml | 44.1 | μg/ml | >100 | μg/ml | >2.27 |
| Strain Long | Ribavirin | 100 | μg/ml | 12.2 | μg/ml | >100 | μg/ml | >8.20 |
| Coxsackie A | Fucoidan | 100 | μg/ml | 7.92 | μg/ml | >100 | μg/ml | >12.6 |
| Strain A7 | Ribavirin | 100 | μg/ml | 7.04 | μg/ml | >100 | μg/ml | >14.2 |
| Rhinovirus | Fucoidan | 100 | μg/ml | 19.1 | μg/ml | >100 | μg/ml | >5.24 |
| Strain 1B | Enviroxime | 10 | μg/ml | 0.485 | μg/ml | >10 | μg/ml | >20.6 |
| HIV-1 | Fucoidan | 100 | μg/ml | 5.24 | μg/ml | >100 | μg/ml | >19.1 |
| Strain IIIB | AZT | 1,000 | nM | 172 | nM | >1,000 | nM | >5.83 |

N/A—Not applicable

### DISCUSSION

The anti-viral activity of Macrocystis pyrifera derived High-Purity Fucoidan demonstrated in the in vitro test results set forth above suggests that the present inventors may have achieved the long-felt, yet unresolved need in the art for finding a cure to the world's HIV/AIDS epidemic as well as providing an economically feasible prevention, treatment, and cure for many thought un-curable and oft untreatable viruses. The demonstrable results show the incredible and unexpected results of Macrocystis pyrifera derived High-Purity Fucoidan in comparison to the best known agents for treating the viruses identified above. The present inventors believe other physiologically similar viruses may also be treatable with the Macrocystis pyrifera derived High-Purity Fucoidan of the present invention. In addition, the present inventors believe the health benefits discussed herein may also be adapted from the Kelp Oil and Kelp Concentrate of Macrocystis pyrifera.

In operation, the Macrocystis pyrifera Fucoidan (or Kelp Concentrate or Kelp Oil) is supplied in an effective amount by a suitable means of delivery to a patient (e.g. oral, transdermal, topical, injection or with adjuvant) to treat or prevent viral infection. It is well within the skill of one of ordinary skill in the art armed with the present specification to determine effective amounts and suitable means through ordinary, routine experimentation.

According to another embodiment, the antiviral protection is acquired over time through the regular, repeated ingestion or intake of an effective dosage regimen of the Fucoidan, Kelp Concentrate and/or Kelp Oil.

### III CONCLUSION

As clear from the above, the Macrocystis pyrifera derived High-Purity Fucoidan and its Kelp Oil and Kelp Concentrate of the present invention provide health benefits never before thought achievable and in a manner that is at odds with conventional wisdom and the credulity of those of ordinary skill in the art.

The above described embodiments were for illustrative purposes of presently preferred embodiments. However, one of ordinary skill in the art armed with the present specification should readily appreciate that the superior and unexpected results of the present invention may provide benefits and find uses in conjunction with similar or other products, features, uses and processes and extrapolated to other products, fields, and combinations.

In sum, those skilled in the art will appreciate that various adaptations and modifications of the above-described preferred embodiments can be configured without departing from the scope and spirit of the invention. Therefore, it is to be understood that the invention may be practiced other than as specifically described herein.

We claim:

1. A method of making a health supplement comprising:
harvesting macroalgae from a body of water; and
collecting macroalgae exudate from the harvested kelp.

2. The method of claim 1 further comprising the step of drying the exudate to form a generally dry solid residue.

3. The method of claim 1 wherein said macroalgae is a species of brown macroalgae.

4. The method of claim 3 wherein said brown macroalagae is Macrocystis pyrifera.

5. The method of claim 2 wherein said macroalgae is a species of brown macroalgae.

6. The method of claim 5 wherein said brown macroalgae is Macrocystis pyrifera.

7. The method of claim 1 further comprising the step of blending the exudate with krill oil.

8. The method of claim 7, wherein said composition comprises about 50% exudate and 50% krill oil.

9. The method of claim 2 further comprising the step of blending the solid residue with krill oil.

10. The method of claim 9 wherein said composition comprises about 50% solid residue and 50% krill oil.

11. The method of claim 1 further comprising the steps of chopping and milling the harvested macroalgae;
Gravitationally draining exudate from said chopped and milled macroalgae;
Collecting drained exudate and mixing with said collected exudate to make a kelp oil mixture;
Sterilizing said kelp oil mixture;
Spray drying said kelp oil mixture;
And collecting said dried kelp oil mixture.

12. The method of claim 11 wherein said collecting steps occur within 12 to 24 hours of said harvesting step.

13. The method of claim 12 further comprising the step of adding said dried kelp oil mixture to a consumer product safe for oral consumption.

14. A health supplement comprising an effective amount of a solid residue material made by harvesting macroalgae, collecting at least a portion of said macroalgae's exudate, and drying said exudate.

15. The health supplement of claim 14 wherein said exudate is collected within 12 to 24 hours of harvesting said macroalgae.

16. The health supplement of claim 15 further comprising the step of adding krill oil in an amount about equal to a predetermined amount of said dried exudate.

17. The health supplement of claim 15 further comprising the step of adding Fucoidan derived from Macrocystis pyrifera in an amount about equal to a predetermined amount of said dried exudate.

18. A method of treating or preventing a retrovirus comprising the steps of administering an effective amount of High Purity Fucoidan derived from Macrocystis pyrifera.

19. The method of claim 18 wherein said retrovirus is selected from the group consisting of DENV2, HSV-1, Influenza A, RSV, Coxsackie A, Rhinovirus and HIV-1.

20. The method of claim 19 wherein said administering step comprising oral, topical, transdermal, injection, or adjuvant administration in a dosing regimen sufficient to treat or prevent infection.
